(19) United States
(12) Patent Application Publication
(10) Pub. No.: US 2024/0228970 A1
(43) Pub. Date: Jul. 11, 2024

(54) METHODS AND SYSTEMS FOR THE SCALABLE GROWTH AND MAINTENANCE OF STEM CELLS USING OPTOGENETICS IN SUSPENSION

(71) Applicant: Prolific Machines Inc., Emeryville, CA (US)

(72) Inventors: Deniz KENT, Oakland, CA (US); Maximiliaan HUISMAN, Oakland, CA (US); Declan JONES, Oakland, CA (US); Victor JONES, Berkeley, CA (US); Monique HOOLEY, Oakland, CA (US); Emily GALE, Berkeley, CA (US); William C. PATON, San Francisco, CA (US)

(21) Appl. No.: 18/417,480

(22) Filed: Jan. 19, 2024

Related U.S. Application Data
(63) Continuation of application No. PCT/US2022/037854, filed on Jul. 21, 2022.
(60) Provisional application No. 63/224,178, filed on Jul. 21, 2021.

(51) Int. Cl. C12N 5/0775; C12M 1/00

(52) U.S. Cl. CPC C12N 5/0662; C12M 31/00; C12N 5/0667; C12N 2501/115; C12N 2510/00; C12N 2529/10

(57) ABSTRACT

Provided herein are methods and systems for growing and maintaining stem cells in suspension. In some cases, the methods and systems involve the use of optical switches for activating one or more growth factor signaling pathways involved in the growth and/or maintenance of stem cells.

### CROSS-REFERENCE

This application is a continuation application of International Patent Application No. PCT/US2022/037854, filed Jul. 21, 2022, which claims the benefit of U.S. Provisional Application No. 63/224,178, filed Jul. 21, 2021, each of which is incorporated herein by reference in its entirety.

### BACKGROUND

Cell production is currently required at a scale not seen before, with demand set to increase dramatically. Of particular interest is the production of stem cells; however, control of the processes that maintain stemness is complex. Stem cells generally require one or more protein factors (e.g., growth factors) present in the culture media to promote growth and/or maintenance (of stemness). Successfully culturing these cells requires tightly-controlled combinations and proportions of growth factors. However, these growth factors are extremely expensive to produce, making large-scale growth and maintenance of stem cells prohibitively expensive. In addition, two-dimensional culture of stem cells (e.g., adherent stem cells, e.g., grown on tissue culture plates) is limited by the surface area of the culture vessel. Thus, scalable growth and maintenance of stem cells is challenging.

### SUMMARY

There is an unmet need for growth and maintenance of stem cells in suspension without the need for using expensive growth factors. The methods and systems disclosed herein use light-activatable domains to activate signaling pathways necessary for growing and maintaining stem cells, thereby meeting this unmet need.

In one aspect, a method of growing and/or maintaining stem cells in suspension is provided, the method comprising: (a) culturing the stem cells in a culture media in suspension, wherein the stem cells have been genetically engineered to express a first fusion protein, the first fusion protein comprising a first signaling protein receptor or a functional portion thereof and a first light-activatable domain; and (b) exposing the stem cells to light thereby activating the first light-activatable domain resulting in activation of a downstream signaling pathway of the first signaling protein receptor, such that the stem cells are grown and/or maintained in suspension. In some cases, the signaling protein receptor or functional portion thereof is a growth factor receptor. In some cases, the culture media is deficient in one or more factor required for growing and/or maintaining the stem cells. In some cases, the one or more factor is a ligand for the first signaling protein receptor or functional portion thereof. In some cases, the one or more factor comprises one or more growth factor. In some cases, the one or more growth factor is transforming growth factor beta (TGFβ). In some cases, the one or more growth factor is a fibroblast growth factor (FGF). In some cases, the FGF is FGF2. In some cases, the first signaling protein receptor or functional portion thereof is a fibroblast growth factor receptor (FGFR) or a transforming growth factor receptor (TGFR). In some cases, the first signaling protein receptor or functional portion thereof is selected from the group consisting of TGFβR1, TGFβR2, FGFR1, FGFR2, and any combination thereof. In some cases, the stem cells are mammalian stem cells. In some cases, the stem cells are human or bovine. In some cases, the stem cells are pluripotent stem cells or multipotent stem cells. In some cases, the pluripotent stem cells are maintained, or are capable of being maintained, in a pluripotent state for at least 7 days. In some cases, the pluripotent stem cells are maintained, or are capable of being maintained, in a pluripotent state for at least 1 month. In some cases, the multipotent stem cells are maintained, or are capable of being maintained, in a multipotent state for at least 7 days. In some cases, the multipotent stem cells are maintained, or are capable of being maintained, in a multipotent state for at least 1 month. In some cases, the stem cells are grown and/or maintained, or are capable of being grown and/or maintained, in suspension for at least 7 days. In some cases, the stem cells are grown and/or maintained, or are capable of being grown and/or maintained, in suspension for at least 1 month. In some cases, the stem cells remain, or are capable of remaining, in an undifferentiated state for at least 7 days. In some cases, the stem cells remain, or are capable of remaining, in an undifferentiated state for at least 1 month. In some cases, the stem cells are embryonic stem cells. In some cases, the stem cells are mesenchymal stem cells. In some cases, the stem cells are satellite cells or muscle stem cells. In some cases, the stem cells are fat stem cells. In some cases, the suspension culture has a volume of at least 100 milliliters (mL). In some cases, the suspension culture is contained within a bioreactor vessel. In some cases, the bioreactor vessel has a total volume of at least 100 milliliters (mL). In some cases, the light-activatable domain is selected from the group consisting of a Light-Oxygen-Voltage (LOV) photoreceptor domain, a LOV2 photoreceptor domain, a Cryptochrome (CRY) photoreceptor domain, Blue-light-using FAD (BLUF) photoreceptor domain, a Phytochrome (PHY) photoreceptor domain, a CIBN (N-terminal domain of CIB1 (cryptochrome-interacting basic-helix-loop-helix protein 1)) domain, a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof. In some cases, the first fusion protein, after the exposing to light of (b), dimerizes or oligomerizes. In some cases, the stem cells are genetically engineered to express a second fusion protein comprising a second signaling protein receptor or functional portion thereof and a second light-activatable domain. In some cases, the second signaling protein receptor or functional portion thereof is different from the first signaling protein receptor or functional portion thereof. In some cases, the second light-activatable domain is different from the first light-activatable domain. In some cases, the first signaling protein receptor or functional portion thereof is TFGβR1 or TGFβR2, and the second signaling protein receptor or functional portion thereof is FGFR1 or FGFR2. In some cases, the downstream signaling pathway is a SMAD2/3 signaling pathway. In some cases, the downstream signaling pathway is an ERK signaling pathway. In some cases, the first signaling protein receptor or functional portion thereof, the second signaling protein receptor or functional portion thereof, or both, does not comprise a fibroblast growth factor receptor (FGFR). In some cases, the exposing of (b) comprises exposing the stem cells to light at a wavelength from 100 nm to 1 mm. In some cases, the exposing of (b) comprises exposing the stem cells to ultraviolet light, visible light, near infrared light, infrared light, or a combination thereof. In some cases, the visible light is blue light, red light, green light, or a combination thereof. In some cases, the exposing of (b) comprises exposing the stem cells to light having one or more illumination parameters. In some cases, the one or more illumination parameters comprises light intensity. In some cases, the one or more illumination parameters comprises a temporal pattern of illumination. In some cases, the temporal pattern comprises a light stimulus duration of at least about one tenth of a second, at least about 1 second, at least about 1 minute, at least about 5 minutes, at least about 30 minutes, or at least about 1 hour. In some cases, the temporal pattern comprises a light stimulus duration of at least about 5 minutes. In some cases, the light stimulus duration is continuous illumination. In some cases, the temporal pattern comprises an interstimulus duration of at least about 1 second, at least about 1 minute, at least about 5 minutes, at least about 10 minutes, at least about 30 minutes, at least about 1 hour, at least about 2 hours, at least about 3 hours, at least about 4 hours, at least about 5 hours, or greater. In some cases, the interstimulus duration is from about 20 minutes to about 250 minutes. In some cases, at 7 days or greater in suspension culture, the stem cells express one or more of markers of stemness.

In another aspect, a system for growing and/or maintaining stem cells is provided, the system comprising: (a) a bioreactor vessel comprising a culture media; (b) a plurality of stem cells in suspension in the culture media, wherein the stem cells have been genetically engineered to express a first fusion protein comprising a first signaling protein receptor or functional portion thereof and a first light-activatable domain; and (c) one or more light source for exposing the stem cells to light to activate the first light-activatable domain resulting in a downstream signaling pathway of the first signaling protein receptor or functional portion thereof, such that the stem cells are grown and/or maintained in suspension. In some cases, the bioreactor vessel has a total volume of at least 100 milliliters (mL). In some cases, the one or more light source comprises one or more light emitting diodes (LEDs). In some cases, the one or more LEDs comprises at least two different LEDs. In some cases, the at least two different LEDs emit light at different wavelengths. In some cases, the one or more light source comprises one or more lasers. In some cases, the one or more light source comprises an incandescent light source. In some cases, the one or more light source is located inside the bioreactor vessel, or located on an interior surface of the bioreactor vessel. In some cases, the one or more light source is located outside the bioreactor vessel, or on an exterior surface of the bioreactor vessel. In some cases, the bioreactor vessel comprises at least one wall or surface that is optically transparent. In some cases, the system further comprises a temperature source for controlling a temperature of the culture media. In some cases, the system further comprises an agitation source for agitating the culture media. In some cases, the system is configured to provide light from the one or more light source in a pattern. In some cases, the pattern is a spatial pattern, a temporal pattern, or both. In some cases, the temporal pattern comprises a light stimulus duration and an interstimulus duration. In some cases, the temporal pattern comprises a light stimulus duration of at least about one tenth of a second, at least about 1 second, at least about 1 minute, at least about 5 minutes, at least about 30 minutes, or at least about 1 hour. In some cases, the light stimulus duration is at least about 5 minutes. In some cases, the temporal pattern comprises an interstimulus duration of at least about 1 second, at least about 1 minute, at least about 5 minutes, at least about 10 minutes, at least about 30 minutes, at least about 1 hour, at least about 2 hours, at least about 3 hours, at least about 4 hours, at least about 5 hours, or greater. In some cases, the interstimulus duration is from about 20 minutes to about 250 minutes. In some cases, the first signaling protein receptor is a growth factor receptor. In some cases, the cell culture media is deficient in one or more factor required for growing and/or maintaining the stem cells. In some cases, the one or more factor is a ligand for the first signaling protein receptor or functional portion thereof. In some cases, the one or more factor comprises one or more growth factor. In some cases, the one or more growth factor is transforming growth factor beta (TGFβ). In some cases, the one or more growth factor is fibroblast growth factor (FGF). In some cases, the first signaling protein receptor or functional portion thereof is a fibroblast growth factor receptor (FGFR) or a transforming growth factor receptor (TGFR). In some cases, the first signaling protein receptor or functional portion thereof is selected from the group consisting of: TGFβR1, TGFβR2, FGFR1, FGFR2, and any combination thereof. In some cases, the stem cells are mammalian stem cells. In some cases, the stem cells are human or bovine. In some cases, the stem cells are pluripotent stem cells or multipotent stem cells. In some cases, the pluripotent stem cells are maintained, or are capable of being maintained, in a pluripotent state for at least 7 days. In some cases, the pluripotent stem cells are maintained, or are capable of being maintained, in a pluripotent state for at least 1 month. In some cases, the multipotent stem cells are maintained, or are capable of being maintained, in a multipotent state for at least 7 days. In some cases, the multipotent stem cells are maintained, or are capable of being maintained, in a multipotent state for at least 1 month. In some cases, the stem cells are grown and/or maintained, or are capable of being grown and/or maintained, in suspension for at least 7 days. In some cases, the stem cells are grown and/or maintained, or are capable of being grown and/or maintained, in suspension for at least 1 month. In some cases, the stem cells remain, or are capable of remaining, in an undifferentiated state for at least 7 days. In some cases, the stem cells remain, or are capable of remaining, in an undifferentiated state for at least 1 month. In some cases, the stem cells are embryonic stem cells. In some cases, the stem cells are mesenchymal stem cells. In some cases, the stem cells are satellite cells or muscle stem cells. In some cases, the stem cells are fat stem cells. In some cases, the culture media has a volume of at least 100 milliliters (mL). In some cases, the light-activatable domain is selected from the group consisting of, a Light-Oxygen-Voltage (LOV) photoreceptor domain, a LOV2 photoreceptor domain, a Cryptochrome (CRY) photoreceptor domain, Blue-light-using FAD (BLUF) photoreceptor domain, a Phytochrome (PHY) photoreceptor domain, a CIBN (N-terminal domain of CIB1 (cryptochrome-interacting basic-helix-loop-helix protein 1)) domain, a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof. In some cases, the first fusion protein, after the exposing to light of (b), dimerizes or oligomerizes. In some cases, the stem cells are genetically engineered to express a second fusion protein comprising a second signaling protein receptor or functional portion thereof and a second light-activatable domain. In some cases, the second signaling protein receptor or functional portion thereof is different from the first signaling protein receptor or functional portion thereof. In some cases, the second light-activatable domain is different from the first light-activatable domain. In some cases, the first signaling protein receptor or functional portion thereof is TFGbR1 or TGFβR2, and the second signaling protein receptor or functional portion thereof is FGFR1 or FGFR2. In some cases, the signaling pathway is a SMAD2/3 signaling pathway. In some cases, the downstream signaling pathway is an ERK signaling pathway. In some cases, the first signaling protein receptor or functional portion thereof, the second signaling protein receptor or functional portion thereof, or both, does not comprise a fibroblast growth factor receptor (FGFR). In some cases, the exposing of (b) comprises exposing the stem cells to light at a wavelength from 100 nm to 1 mm. In some cases, the exposing of (b) comprises exposing the stem cells to ultraviolet light, visible light, near infrared light, infrared light, or a combination thereof. In some cases, the visible light is blue light, red light, green light, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1E depict experimental data demonstrating activation of FGF signaling by light in stem cells engineered to express a light-activatable domain fused to a signaling protein receptor, according to embodiments provided herein.

FIG. 2 depicts experimental data demonstrating light-controlled growth of adipose stem cells engineered to express a light-activatable domain fused to a signaling protein receptor, according to embodiments provided herein.

FIG. 3 depicts experimental data demonstrating light-controlled growth of adipose stem cells engineered to express a light-activatable domain fused to a signaling protein receptor, according to embodiments provided herein.

FIG. 4 depicts experimental data demonstrating light-controlled maintenance of sternness of adipose stem cells engineered to express a light-activatable domain fused to a signaling protein receptor, according to embodiments provided herein.

FIGS. 5A and 5B depict experimental data demonstrating light-controlled growth of muscle stem cells engineered to express a light-activatable domain fused to a signaling protein receptor, according to embodiments provided herein.

FIG. 6 depicts experimental data demonstrating light-controlled growth of muscle stem cells engineered to express a light-activatable domain fused to a signaling protein receptor, according to embodiments provided herein.

FIGS. 7A-7D depict experimental data demonstrating light-controlled maintenance of stem cells engineered to express a light-activatable domain fused to a signaling protein receptor, according to embodiments provided herein.

### DETAILED DESCRIPTION

Stem cells generally require one or more protein factors (e.g., growth factors) present in the culture media to promote growth and/or maintenance (of stemness). However, these proteins are extremely expensive to produce making large-scale growth and maintenance of stem cells prohibitively expensive. Additionally, two-dimensional culture of adherent stem cells (e.g., on tissue culture plates) is limited by the surface area of the culture vessel. Provided herein are methods and systems for growing and/or maintaining stem cells in suspension. The methods and systems provided herein generally relate to the use of light-activatable domains fused to a signaling protein receptor (or functional domain or functional portion thereof) for activating downstream signaling pathways necessary for growing and/or maintaining stem cells in suspension. Advantageously, the methods and systems provided herein allow for the growth and maintenance of stem cells in suspension without the need of cost-prohibitive growth factors present in the culture medium. Accordingly, the methods and systems provided herein improve the cost effectiveness and scalability of growing and maintaining stem cells.

### Methods

Provided herein are methods for growing and/or maintaining stem cells in suspension. In various aspects, the methods comprise culturing the stem cells in a culture media in suspension, wherein the stem cells have been genetically engineered to express a first fusion protein, the first fusion protein comprising a first signaling protein receptor or functional domain or functional portion thereof and a first light-activatable domain; and (b) exposing the stem cells to light thereby activating the first light-activatable domain resulting in activation of a downstream signaling pathway of the first signaling protein, such that the stem cells are grown and/or maintained in suspension.

In various aspects, the methods involve the use of stem cells genetically engineered to express a fusion protein. The fusion protein may comprise a light-activatable domain fused to a signaling protein receptor or a portion thereof. The term “light-activatable domain” as used herein refers to a protein or portion thereof that responds to light of a particular wavelength. In some cases, the light-activatable domain, upon stimulation with light of a particular wavelength or within a particular spectral range, dimerizes or oligomerizes (e.g., with another light-activatable domain). In some cases, the light-activatable domain may form a homodimer or a heterodimer (e.g., may dimerize with a second, different light-activatable domain). In some cases, the light-activatable domain may exist in a (e.g., homo or hetero) dimer or (e.g., homo or hetero) oligomer (e.g., in the absence of light), and may dissociate into a monomeric form after exposure to light.

The light-activatable domain may be derived from a natural source (e.g., a naturally-occurring protein) or may be synthetically produced. The light-activatable domain may comprise or may be a functional domain or portion of a naturally occurring protein, such as, by way of example only, the PHR domain of Arabidopsis cryptochrome 2. The light-activatable domain may comprise an amino acid sequence identical to an amino acid sequence of a wild-type protein, or may comprise one or more variants (e.g., amino acid substitutions, deletions, insertions, etc.) relative to a wild-type protein. The light-activatable domain may comprise an amino acid sequence having at least about 50% sequence identity to a naturally-occurring protein (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater).

In various aspects, a combination of light-activatable domains (e.g., a first light-activatable domain and a second light-activatable domain) may be used. In this scenario, the first light-activatable domain and the second light-activatable domain are binding partners, such that upon illumination with light at a particular wavelength or within a particular spectral range, the first and second light-activatable domains heterodimerize or hetero-oligomerize. The first and second light-activatable domains, in this scenario, may be fused to separate signaling protein receptors (or functional domains or functional portions thereof). In some cases, the separate signaling protein receptors (or functional domains or functional portions thereof) are different signaling protein receptors (or functional domains or functional portions thereof) that heterodimerize or hetero-oligomerize. In other cases, the separate signaling protein receptors (or functional domains or functional portions thereof) are the same signaling protein receptor (or functional domain or functional portion thereof) that homodimerize or homo-oligomerize. The first and second light-activatable domains, upon illumination with light at a particular wavelength or within a particular spectral range, heterodimerize or hetero-oligomerize, thereby bringing the signaling protein receptors (or functional domains or functional portions thereof) into close contact with one another such that the corresponding downstream signaling pathway(s) is/are activated.

In various aspects, the light-activatable domain comprises a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, a CIBN (N-terminal domain of CIB1 (cryptochrome-interacting basic-helix-loop-helix protein 1)) domain, a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof. In one example, the light-activatable domain is a LOV domain (e.g., such as a LOV domain derived from Vaucheria frigida Aureochrome1).

In some instances, a combination of light-activatable domains is used, wherein the first light-activatable domain is cryptochrome 2 (or a variant or a functional portion thereof) and the second-light activatable domain is CIBN (or a variant or a functional portion thereof). In some instances, a combination of light-activatable domains is used, wherein the first light-activatable domain is BphP1 (or a variant or a functional portion thereof) and the second-light activatable domain is QPAS1 (or a variant or a functional portion thereof). In some cases, the light-activatable domain (or combination of light-activatable domains) is selected from Table 1. In some cases, the light-activatable domain may have an amino acid sequence having at least 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to any one of the light-activatable domains described in Table 1.

TABLE 1
Non-limiting examples of light-activatable domain systems

| | Binding | Binding | Binding | |
|---|---|---|---|---|
| System | type | partner 1 | partner 2 | Light |
| Light-Oxygen- | Homodimer | VfAU1-LOV | — | Blue light results |
| Voltage (LOV) | | | | in dimerization |
| Cryptochrome | Homodimer/ | Photolyase- | — | Blue light results |
| (CRY) | oligomer | Homologous | | in dimerization |
| | | Region (PHR) | | |
| | | domain | | |
| CRY-CIBN | heterodimer | PHR domain | CIBN | Blue light results |
| | | | | in dimerization |
| Blue-light- | homodimer | BLUF | — | Blue light results |
| using FAD | | domain | | in dimerization |
| (BLUF) | | | | |
| photoreceptor | | | | |
| domain | | | | |
| PhyB/PIF | heterodimer | PhyB | PIF | Red light results in |
| | | | (PIF3, | dimerization, far |
| | | | PIF6) | red light results in |
| | | | | dissociation |
| Dronpa | homodimer | Dronpa | — | Illumination with |
| | | | | blue light results |
| | | | | in |
| | | | | monomerization; |
| | | | | illumination with |
| | | | | UV light results in |
| | | | | dimerization |
| UVR8 | homodimer | UVR8 | — | Dissociates when |
| | | | | exposed to UV-B |
| | | | | light |
| UVR8 | heterodimer | UVR8 | COP1 | UVR8 homodimer |
| | | | | dissociates when |
| | | | | exposed to UV-B |
| | | | | light and forms |
| | | | | heterodimer with |
| | | | | COP1 |
| BphP1/QPAS1 | heterodimer | BphP1 | QPAS-1 | Near infrared light |
| | | | | results in |
| | | | | dimerization; red |
| | | | | light dissociates |
| Cobalamin | oligomer | CBD | — | Homooligomerizes |
| binding | | | | in dark; green light |
| domains | | | | results in |
| (CBD) | | | | dissociation |

In various aspects, the methods involve exposing the stem cells (e.g., genetically engineered to express the fusion protein comprising the signaling protein receptor and the light-activatable domain) with light at a particular wavelength or light within a particular spectral range. The wavelength of light is selected such that the light is capable of activating the light-activatable domain. For example, Table 1 provides non-limiting examples of light parameters for different light-activatable domain systems. The wavelength of light may be one or more of infrared, near infrared, visible light (e.g., red, green, blue), ultraviolet light, or a combination thereof. Infrared light may comprise light at a wavelength of about 780 nm to 1 mm. Near infrared light may comprise light at a wavelength of about 740 nm to about 780 nm. Red light may comprise light at a wavelength of about 620 nm to 750 nm, 600 nm to 690 nm, or about 650 nm. Green light may comprise light at a wavelength of about 577 nm to about 492 nm. Blue light may comprise light at a wavelength of 492 to about 455 nm, or about 440 nm to about 473 nm. Ultraviolet light may comprise light from about 10 nm to 400 nm, or from about 280 to 315 nm. In various aspects, the wavelength of light is from 100 nm to 1 mm.

In various aspects, the methods involve illuminating the stem cells with light having one or more illumination parameters. In some cases, the one or more illumination parameters includes light intensity and/or a temporal pattern of illumination. In some cases, the temporal pattern may include a stimulus duration and an interstimulus duration. In some cases, the temporal pattern comprises a light stimulus duration of at least about one tenth of a second, at least about 1 second, at least about 1 minute, at least about 5 minutes, at least about 30 minutes, or at least about 1 hour. In some cases, the stimulus duration may be at least about 5 minutes. In some cases, the temporal pattern comprises an interstimulus duration of at least about 1 second, at least about 1 minute, at least about 5 minutes, at least about 10 minutes, at least about 30 minutes, at least about 1 hour, at least about 2 hours, at least about 3 hours, at least about 4 hours, at least about 5 hours, or greater. In some cases, the interstimulus duration may be from about 20 minutes to about 250 minutes.

In various aspects, the light-activatable domain is fused to a signaling protein receptor or a functional portion thereof. The signaling protein receptor is generally a protein receptor that is involved in activating one or more downstream signaling pathways necessary for the growth and/or maintenance of stem cells. The signaling protein receptor may be a protein receptor that requires dimerization or oligomerization in order to activate downstream signaling pathways. Upon illumination of the stem cell by light of an appropriate wavelength, the light-activatable domain dimerizes or oligomerizes, thereby causing dimerization or oligomerization of the signaling protein receptor such that the downstream signaling pathway is activated. In an alternative embodiment, the light-activatable domain may dimerize or oligomerize in the absence of light, and may dissociate (e.g., into monomeric form) upon exposure to light. In this scenario, exposure to light may switch off the downstream signaling pathway (e.g., by dissociating the signaling protein receptors).

In some aspects, a first light-activatable domain is fused to a first signaling protein receptor and a second light-activatable domain is fused to a second signaling protein receptor. In this scenario, the first and second light-activatable domains are binding partners such that, upon illumination with light at a particular wavelength or within a particular spectral range, dimerize or oligomerize (e.g., as described herein). Additionally, the first signaling protein receptor and the second signaling protein receptor dimerize or oligomerize (upon dimerization or oligomerization of the first and second light-activatable domains). When the first and second signaling protein receptors are in close contact, downstream signaling pathways associated with these signaling protein receptors are activated.

In another aspect, a first light-activatable domain is fused to a first signaling protein receptor and a second light-activatable domain is fused to a second signaling protein receptor. In this scenario, the first and second light-activatable domains are binding partners such that they dimerize or oligomerize (e.g., as described herein) in the absence of light. Additionally, the first signaling protein receptor and the second signaling protein receptor dimerize or oligomerize (upon dimerization or oligomerization of the first and second light-activatable domains). When the first and second signaling protein receptors are in close contact, downstream signaling pathways associated with these signaling protein receptors are activated. In this scenario, exposure to light causes the first and second light-activatable domains to dissociate (into monomeric form), thereby removing the contact between the first and second signaling protein receptors, and deactivating the downstream signaling pathway.

Various growth factors may be used in a cell culture media to grow and maintain stem cells in vitro. These growth factors include, without limitation, a fibroblast growth factor (FGF), transforming growth factor beta (TGFβ), activin, Nodal, and LIF. In various aspects, the methods disclosed herein provide for growing and/or maintaining stem cells in a culture media in the absence of or deficient for one or more of a fibroblast growth factor (FGF), transforming growth factor beta (TGFβ), activin, Nodal, and LIF. In various aspects, the signaling protein receptor is a protein receptor that is activated by one or more of these growth factors.

In various aspects, the signaling protein receptor is a transforming growth factor beta receptor (TGFβR). In some cases, the TFGβR may be one or more of TGFβR1, TGFβR2, or TGFβR3. In some cases, the signaling protein receptor is a protein receptor that normally dimerizes or oligomerizes upon binding of one or more ligands selected from TGFβ1, TGFβ2, or TGFβ3. In some cases, the signaling protein receptor may be a variant (e.g., comprising one or more amino acid substitutions, insertions, deletions, and the like) of TGFβR1, TGFβR2, or TGFβR3. In some cases, the signaling protein receptor may be a functional domain or a functional portion of TGFβR1, TGFβR2, or TGFβR3. In some cases, the signaling protein receptor may have an amino acid sequence having at least 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to a wild-type TGFβR1, TGFβR2, or TGFβR3 amino acid sequence.

In various aspects of the disclosure, the methods involve the growth and/or maintenance of stem cells in a culture media absent of or deficient for one or more of TGFβ1, TGFβ2, or TGFβ3 (e.g., by using a fusion protein comprising TGFβR or a functional portion thereof and a light-activatable domain, e.g., as disclosed herein). In various aspects, illumination of stem cells (e.g., genetically engineered to express a fusion protein comprising TGFβR or a functional portion thereof and a light-activatable domain) with light results in dimerization or oligomerization of TGFβR. In various aspects, illumination of stem cells (e.g., genetically engineered to express a fusion protein comprising TGFβR or a functional portion thereof and a light-activatable domain) with light results in activation of one or more downstream signaling pathways associated with TGFβR. In some cases, the one or more downstream signaling pathways associated with TGFβR is a SMAD2/3 signaling pathway.

In various aspects, the signaling protein receptor is a fibroblast growth factor receptor (FGFR). In some cases, the FGFR may be one or more of FGFR1, FGFR2, FGFR3, FGFR4, FGFRL1, or FGFR6. In some cases, the signaling protein receptor is a protein receptor that normally dimerizes or oligomerizes upon binding of one or more ligands selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23. In various aspects of the disclosure, the methods involve the growth and/or maintenance of stem cells in a culture media absent of or deficient for one or more of FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 (e.g., by using a fusion protein comprising FGFR or a functional portion thereof and a light-activatable domain, e.g., as disclosed herein). In some cases, the signaling protein receptor may be a variant (e.g., comprising one or more amino acid substitutions, insertions, deletions, and the like) of FGFR1, FGFR2, FGFR3, FGFR4, FGFRL1, or FGFR6. In some cases, the signaling protein receptor may be a functional domain or a functional portion of FGFR1, FGFR2, FGFR3, FGFR4, FGFRL1, or FGFR6. In some cases, the signaling protein receptor may have an amino acid sequence having at least 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to a wild-type FGFR1, FGFR2, FGFR3, FGFR4, FGFRL1, or FGFR6 amino acid sequence.

In various aspects, illumination of stem cells (e.g., genetically engineered to express a fusion protein comprising FGFR or a functional portion thereof and a light-activatable domain) with light results in dimerization or oligomerization of FGFR. In various aspects, illumination of stem cells (e.g., genetically engineered to express a fusion protein comprising FGFR or a functional portion thereof and a light-activatable domain) with light results in activation of one or more downstream signaling pathways associated with FGFR. In some cases, the one or more downstream signaling pathways associated with FGFR is an ERK signaling pathway. In some cases, the signaling protein receptor does not comprise FGFR.

In various aspects, the stem cells may be genetically engineered to express a first fusion protein comprising a first signaling protein receptor and a first light-activatable domain (activatable by light at a first wavelength or within a first spectral range), and a second fusion protein comprising a second signaling protein receptor and a second light-activatable domain (activatable by light at a second wavelength or within a second spectral range). In such cases, the first signaling protein receptor and the second signaling protein receptor are different protein receptors, each capable of activating a downstream signaling pathway important or necessary for growing and/or maintaining stem cells. Furthermore, the first light-activatable domain and the second light-activatable domain are different, and activatable by light at different wavelengths or within different spectral ranges. Such scenarios allow for the precise control of signaling pathway activation, and allow the user to modulate one signaling pathway without affecting the other. In a non-limiting example, the first signaling protein receptor is FGFR wherein, upon illumination of light at a first wavelength or within a first spectral range, the FGFR dimerizes or oligomerizes thereby activating a first signaling pathway (e.g., an ERK signaling pathway); and the second signaling protein receptor is TGFβR wherein, upon illumination of light at a second, different wavelength or within a second, different spectral range, the TGFβR dimerizes or oligomerizes thereby activating a second signaling pathway (e.g., SMAD2/3 signaling pathway).

The stem cells used in the methods provided herein may be any desired stem cell. In some instances, the stem cells are pluripotent stem cells. In some instances, the stem cells are multipotent stem cells. In some cases, the stem cells are embryonic stem cells. In some cases, the stem cells are mesenchymal stem cells. In some cases, the stem cells are satellite cells or muscle stem cells. In some cases, the stem cells are fat stem cells. In certain embodiments, the stem cells described herein are mammalian stem cells. In some cases, the mammalian stem cells are selected from the group consisting of: human stem cells, bovine (cow) stem cells, ovine (sheep) stem cells, and porcine (pig) stem cells. In one example, the mammalian stem cells are bovine stem cells. In some cases, the bovine stem cells are derived from a Wagyu bull or an Angus bull. In some cases, the stem cells are avian stem cells, such as, but not limited to, chicken stem cells. In some cases, the stem cells are fish stem cells, such as, but not limited to, tuna stem cells.

Advantageously, the methods herein provide for growing and/or maintaining stem cells in suspension (rather than as, e.g., two-dimensional, adherent cell cultures). In some cases, the methods provided herein do not require the use of a feeder layer of cells. In some cases, the methods provided herein do not require the use of extracellular matrix components. In some cases, the methods involve growing or maintaining stem cells in a bioreactor. In some cases, the methods provided herein may involve the use of microcarriers (e.g., beads). In some cases, microcarriers may be coated with extracellular matrix components (e.g., to promote attachment of stem cells thereto).

In various aspects, the suspension culture has a volume of at least about 100 milliliters (mL). For example, the suspension culture may have a volume of at least about 150 mL, at least about 200 mL, at least about 250 mL, at least about 300 mL, at least about 350 mL, at least about 400 mL, at least about 450 mL, at least about 500 mL, at least about 550 mL, at least about 600 mL, at least about 650 mL, at least about 700 mL, at least about 750 mL, at least about 800 mL, at least about 850 mL, at least about 900 mL, at least about 950 mL, at least about 1000 mL, at least about 2000 mL, at least about 3000 mL, at least about 4000 mL, or at least about 5000 mL. In some cases, the suspension culture has a volume of less than about 1000 mL. In some cases, the suspension culture has a volume of greater than about 1000 mL.

Any suitable cell culture media for growing and/or maintaining stem cells may be used. In some embodiments, the cell culture media comprises any of the following in an appropriate combination: isotonic saline, buffer, amino acids, serum or serum replacement, sugars (e.g., glucose), and other exogenously added factors. In some embodiments, the cell culture media comprises DMEM, F12, aMEM, Hepatostim™, RPMI, or combinations thereof, either in the presence or absence or serum. Suitable sera include calf serum, fetal calf serum, horse serum, or the like. In some embodiments, a serum supplement is used.

In some embodiments, the cell culture media is deficient in one or more factor required for growing and/or maintaining the stem cells. In some embodiments, the cell culture media is deficient for FGF, TGFβ, or both. In some embodiments, the cell culture media is deficient in FGF2, TGFβ1, activin, Nodal, LIF, or a combination thereof.

The fusion proteins described herein can be encoded by a nucleic acid. In some embodiments, a nucleic acid comprising the fusion protein can be an expression cassette or can be comprised within an expression cassette. As used herein, “expression cassette” means a recombinant nucleic acid construct comprising one or more nucleic acids described herein, wherein the recombinant nucleic acid construct is operably associated with at least one control sequence (e.g., a promoter).

In certain embodiments, the nucleic acid is a component of a vector that can be used to transfer the nucleic acid into a cell. As used herein, the term “vector” refers to a nucleic acid molecule capable of transporting another nucleic acid to which it has been linked. One type of vector is a genomic integration vector, or “integration vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors”>Suitable vectors comprise plasmids, bacterial artificial chromosomes, yeast artificial chromosomes, viral vectors and the like.

In the vectors, regulatory elements such as promoters, enhancers, and polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

In some aspects, the nucleic acids that are introduced into a eukaryotic cell are operably linked to a promoter and/or to a polyA signal as known in the art. In some embodiments, the nucleic acids having a 5′ end and a 3′ end is operably linked at the 5′ end to a promoter and at the 3′ end to a polyA signal. In some aspects, the nucleic acids comprise 2A peptide sequences and/or internal ribosomal entry sites.

In some embodiments, the expression cassette includes a nucleotide sequence encoding a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence).

In various aspects, the methods described herein allow for the stem cells to be maintained in a pluripotent state for an extended period of time. A pluripotent cell can give rise to all cell types within a body. For instance, an embryonic stem cell is capable of differentiating into any cell type within the embryo. In some embodiments, the stem cells are capable of being maintained in a pluripotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are capable of being maintained in a pluripotent state for at least 7 days. In some cases, the stem cells are capable of being maintained in a pluripotent state for at least 1 month. In some embodiments, the stem cells are maintained in a pluripotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are maintained in a pluripotent state for at least 7 days. In some cases, the stem cells are maintained in a pluripotent state for at least 1 month. In various aspects, the stem cells, after performing the methods provided herein, express one or more markers of pluripotency (e.g., OCT4).

In various aspects, the methods described herein allow for the stem cells to be maintained in a multipotent state for an extended period of time. A multipotent stem cell is capable of giving rise to several different cell types. For instance, a mesenchymal stem cell is capable of differentiating into multiple cell types including bone, cartilage, muscle cells, fat cells, and connective tissue. In some embodiments, the stem cells are capable of being maintained in a multipotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are capable of being maintained in a multipotent state for at least 7 days. In some cases, the stem cells are capable of being maintained in a multipotent state for at least 1 month. In some embodiments, the stem cells are maintained in a multipotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are maintained in a multipotent state for at least 7 days. In some cases, the stem cells are maintained in a multipotent state for at least 1 month. In various aspects, the stem cells, after performing the methods provided herein, express one or more markers of multipotency.

In various aspects, the stem cells are capable of being maintained in an undifferentiated state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are capable of being maintained in an undifferentiated state for at least 7 days. In some cases, the stem cells are capable of being maintained in an undifferentiated state for at least 1 month. In various aspects, the stem cells are maintained in an undifferentiated state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are maintained in an undifferentiated state for at least 7 days. In some cases, the stem cells are maintained in an undifferentiated state for at least 1 month. In various aspects, the stem cells, after performing the methods provided herein, do not express one or more markers of differentiation. In various aspects, the stem cells, after performing the methods provided herein, do not express one or more cell or tissue-type specific markers. In various aspects, the stem cells, after performing the methods provided herein, express one or more markers of sternness. In various aspects, the stem cells, after performing the methods provided herein, express one or more markers of an undifferentiated state.

In various aspects, the methods provided herein promote the growth of stem cells in suspension. In some cases, promoting the growth of stem cells comprises increasing proliferation rates. In some cases, promoting the growth of stem cells comprises maintaining proliferation rates.

### Systems

Further described herein are systems for growing and/or maintaining stem cells in suspension. In one aspect, the system comprises (a) a bioreactor vessel comprising a culture media; (b) a plurality of stem cells in suspension in the culture media, wherein the stem cells have been genetically engineered to express a first fusion protein comprising a first signaling protein or portion thereof and a first light-activatable domain; and (c) one or more light source for exposing the stem cells to light to activate the first light-activatable domain resulting in a downstream signaling pathway of the first signaling protein or portion thereof, such that the stem cells are grown and/or maintained in suspension.

The bioreactor may be any type of culture vessel suitable for growing cells in a suspension culture. In various aspects, the bioreactor comprises a volume of at least about 50 mL, at least about 100 mL, at least about 150 mL, at least about 200 mL, at least about 250 mL, at least about 300 mL, at least about 400 mL, at least about 500 mL, at least about 600 mL, at least about 700 mL, at least about 750 mL, at least about 800 mL, at least about 900 mL, at least about 1000 mL, at least about 2000 mL, at least about 3000 mL, at least about 5000 mL, or more.

In various aspects, the bioreactor comprises one or more light source configured to illuminate one or more stem cells. The wavelength of light may be one or more of infrared, near infrared, visible light (e.g., red, green, blue), ultraviolet light, or a combination thereof. Infrared light may comprise light at a wavelength of about 780 nm to 1 mm. Near infrared light may comprise light at a wavelength of about 740 nm to about 780 nm. Red light may comprise light at a wavelength of about 620 nm to 750 nm, 600 nm to 690 nm, or about 650 nm. Green light may comprise light at a wavelength of about 577 nm to about 492 nm. Blue light may comprise light at a wavelength of 492 to about 455 nm, or about 440 nm to about 473 nm. Ultraviolet light may comprise light from about 10 nm to 400 nm, or from about 280 to 315 nm. In various aspects, the wavelength of light is from 100 nm to 1 mm.

In certain embodiments, the one or more light source is within an interior of the bioreactor (e.g., the one or more light source is located on one or more components within the interior of the bioreactor). In some cases, the one or more light source is located on an interior surface of the bioreactor. In certain embodiments, the light source is located on the exterior of the bioreactor. In certain embodiments, the bioreactor comprises an optically transparent surface. In some instances, a light guide may be applied to deliver the light to the cell suspension.

In certain embodiments, the one or more light source comprises at least one light-emitting diode (LED). In certain embodiments, the one or more light source comprises at least one, two, three, four, five, six, seven, eight, nine, ten or more than ten LEDs. In certain embodiments, the one or more light source comprises at least one laser. In some embodiments, the one or more light source comprises at least one, two, three, four, five, six, seven, eight, nine, ten or more than ten lasers. In some embodiments, the one or more light source is configured to emit at least one, two, three, four, five, or more than five different wavelengths of light. In some embodiments, the one or more light source comprises an incandescent light source. In some embodiments, the one or more light source comprises at least one, two, three, four, five, six, seven, eight, nine, ten, or more than ten incandescent light sources. The one or more light source may be configured to provide light to the system in a pattern (e.g., a spatial pattern, a temporal pattern, or both). The temporal pattern may include a stimulus duration and an interstimulus duration. In some cases, the temporal pattern comprises a light stimulus duration of at least about one tenth of a second, at least about 1 second, at least about 1 minute, at least about 5 minutes, at least about 30 minutes, or at least about 1 hour. In some cases, the stimulus duration (e.g., the amount of time the cells are exposed to light) may be at least about 5 minutes. In some cases, the temporal pattern comprises an interstimulus duration of at least about 1 second, at least about 1 minute, at least about 5 minutes, at least about 10 minutes, at least about 30 minutes, at least about 1 hour, at least about 2 hours, at least about 3 hours, at least about 4 hours, at least about 5 hours, or greater. In some cases, the interstimulus duration (e.g., the amount of time between periods of illumination) may be from about 20 minutes to about 250 minutes.

In certain embodiments, the bioreactor comprises a system for regulation of temperature (e.g., for regulation the temperature of the culture media). The temperature may be selected to be suitable for culturing stem cells. In certain embodiments, the bioreactor comprises an agitator (e.g., for agitating the culture media). In some embodiments, the bioreactor comprises a sensor. In some embodiments, the sensor comprises a sensor that detects optical density, temperature, CO2 levels, liquid level, pH, oxygen levels, color, rotational speed (e.g., of the agitator) or a combination thereof.

In various aspects, the systems comprise stem cells genetically engineered to express a fusion protein. The fusion protein may comprise a light-activatable domain fused to a signaling protein receptor or a portion thereof (e.g., as described herein). In some cases, the light-activatable domain, upon stimulation with light of a particular wavelength, dimerizes or oligomerizes (e.g., with another light-activatable domain). In some cases, the light-activatable domain may form a homodimer or a heterodimer (e.g., may dimerize with a second, different light-activatable domain).

The light-activatable domain may be derived from a natural source (e.g., a naturally-occurring protein) or may be synthetically produced. The light-activatable domain may comprise or may be a functional domain or portion of a naturally occurring protein. The light-activatable domain may comprise an amino acid sequence identical to an amino acid sequence of a wild-type protein, or may comprise one or more variants (e.g., amino acid substitutions, deletions, insertions, etc.) relative to a wild-type protein. The light-activatable domain may comprise an amino acid sequence having at least about 50% sequence identity to a naturally-occurring protein (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater).

In various aspects, the light activatable domain comprises a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, a CIBN (N-terminal domain of CIB1 (cryptochrome-interacting basic-helix-loop-helix protein 1)) domain, a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof. In one example, the light-activatable domain is a LOV domain (such as an LOV domain from Vaucheria frigida Aureochrome1).

In some instances, a combination of light-activatable domains is used, wherein the first light-activatable domain is cryptochrome 2 (or a variant or a functional portion thereof) and the second-light activatable domain is CIBN (or a variant or a functional portion thereof). In some instances, a combination of light-activatable domains is used, wherein the first light-activatable domain is BphP1 (or a variant or a functional portion thereof) and the second-light activatable domain is QPAS1 (or a variant or a functional portion thereof). In some cases, the light-activatable domain (or combination of light-activatable domains) is selected from Table 1. In some cases, the light-activatable domain may have an amino acid sequence having at least 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to any one of the light-activatable domains described in Table 1.

In various aspects, the system is configured to expose the stem cells (e.g., genetically engineered to express the fusion protein comprising the signaling protein receptor and the light-activatable domain) to light at a particular wavelength or within a particular spectral range. The wavelength of light or spectral range is selected such that the light is capable of activating the light-activatable domain. For example, Table 1 provides non-limiting examples of light parameters for different light-activatable domain systems. The wavelength of light may be one or more of infrared, near infrared, visible light (e.g., red, green, blue), ultraviolet light, or a combination thereof. Infrared light may comprise light at a wavelength of about 780 nm to 1 mm. Near infrared light may comprise light at a wavelength of about 740 nm to about 780 nm. Red light may comprise light at a wavelength of about 620 nm to 750 nm, 600 nm to 690 nm, or about 650 nm. Green light may comprise light at a wavelength of about 577 nm to about 492 nm. Blue light may comprise light at a wavelength of 492 to about 455 nm, or about 440 nm to about 473 nm. Ultraviolet light may comprise light from about 10 nm to 400 nm, or from about 280 to 315 nm. In some cases, the wavelength of light is from 100 nm to 1 mm.

In various aspects, the light-activatable domain is fused to a signaling protein receptor or a functional portion thereof. The signaling protein receptor is generally a protein receptor that is involved in activating one or more downstream signaling pathways necessary for the growth and/or maintenance of stem cells. The signaling protein receptor may be a protein receptor that requires dimerization or oligomerization in order to activate downstream signaling pathways. Upon illumination of the stem cell by light of an appropriate wavelength, the light-activatable domain dimerizes or oligomerizes, thereby causing dimerization or oligomerization of the signaling protein receptor such that the downstream signaling pathway is activated.

In some aspects, a first light-activatable domain is fused to a first signaling protein receptor and a second light-activatable domain is fused to a second signaling protein receptor. In this scenario, the first and second light-activatable domains are binding partners such that, upon illumination with light at a particular wavelength or within a particular spectral range, dimerize or oligomerize (e.g., as described herein). Additionally, the first signaling protein receptor and the second signaling protein receptor dimerize or oligomerize (upon dimerization or oligomerization of the first and second light-activatable domains). When the first and second signaling protein receptors are in close contact, downstream signaling pathways associated with these signaling protein receptors are activated.

Various growth factors may be used in a cell culture media to grow and maintain stem cells in vitro. These growth factors include, without limitation, a fibroblast growth factor (FGF), transforming growth factor beta (TGFβ), activin, Nodal, and LIF. In various aspects, the systems disclosed herein provide for growing and/or maintaining stem cells in a culture media in the absence of or deficient for one or more of a fibroblast growth factor (FGF), transforming growth factor beta (TGFβ), activin, Nodal, and LIF. In various aspects, the signaling protein receptor is a protein receptor that is activated by one or more of these growth factors.

In various aspects, the signaling protein receptor is a transforming growth factor beta receptor (TGFβR). In some cases, the TFGβR may be one or more of TGFβR1, TGFβR2, or TGFβR3. In some cases, the signaling protein receptor is a protein receptor that normally dimerizes or oligomerizes upon binding of one or more ligands selected from TGFβ1, TGFβ2, or TGFβ3. In some cases, the signaling protein receptor may be a variant (e.g., comprising one or more amino acid substitutions, insertions, deletions, and the like) of TGFβR1, TGFβR2, or TGFβR3. In some cases, the signaling protein receptor may be a functional domain or a functional portion of TGFβR1, TGFβR2, or TGFβR3. In some cases, the signaling protein receptor may have an amino acid sequence having at least 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to a wild-type TGFβR1, TGFβR2, or TGFβR3 amino acid sequence.

In various aspects of the disclosure, the methods involve the growth and/or maintenance of stem cells in a culture media absent of or deficient for one or more of TGFβ1, TGFβ2, or TGFβ3 (e.g., by using a fusion protein comprising TGFβR or a functional portion thereof and a light-activatable domain, e.g., as disclosed herein). In various aspects, illumination of stem cells (e.g., genetically engineered to express a fusion protein comprising TGFβR or a functional portion thereof and a light-activatable domain) with light results in dimerization or oligomerization of TGFβR. In various aspects, illumination of stem cells (e.g., genetically engineered to express a fusion protein comprising TGFβR or a functional portion thereof and a light-activatable domain) with light results in activation of one or more downstream signaling pathways associated with TGFβR. In some cases, the one or more downstream signaling pathways associated with TGFβR is a SMAD2/3 signaling pathway.

In various aspects, the signaling protein receptor is a fibroblast growth factor receptor (FGFR). In some cases, the FGFR may be one or more of FGFR1, FGFR2, FGFR3, FGFR4, FGFRL1, or FGFR6. In some cases, the signaling protein receptor is a protein receptor that normally dimerizes or oligomerizes upon binding of one or more ligands selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23. In various aspects of the disclosure, the methods involve the growth and/or maintenance of stem cells in a culture media absent of or deficient for one or more of FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 (e.g., by using a fusion protein comprising FGFR or a functional portion thereof and a light-activatable domain, e.g., as disclosed herein). In some cases, the signaling protein receptor may be a variant (e.g., comprising one or more amino acid substitutions, insertions, deletions, and the like) of FGFR1, FGFR2, FGFR3, FGFR4, FGFRL1, or FGFR6. In some cases, the signaling protein receptor may be a functional domain or a functional portion of FGFR1, FGFR2, FGFR3, FGFR4, FGFRL1, or FGFR6. In some cases, the signaling protein receptor may have an amino acid sequence having at least 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to a wild-type FGFR1, FGFR2, FGFR3, FGFR4, FGFRL1, or FGFR6 amino acid sequence.

In various aspects, illumination of stem cells (e.g., genetically engineered to express a fusion protein comprising FGFR or a functional portion thereof and a light-activatable domain) with light results in dimerization or oligomerization of FGFR. In various aspects, illumination of stem cells (e.g., genetically engineered to express a fusion protein comprising FGFR or a functional portion thereof and a light-activatable domain) with light results in activation of one or more downstream signaling pathways associated with FGFR. In some cases, the one or more downstream signaling pathways associated with FGFR is an ERK signaling pathway. In some cases, the signaling protein receptor does not comprise FGFR.

In various aspects, the stem cells may be genetically engineered to express a first fusion protein comprising a first signaling protein receptor and a first light-activatable domain (activatable by light at a first wavelength or within a first spectral range), and a second fusion protein comprising a second signaling protein receptor and a second light-activatable domain (activatable by light at a second wavelength or within a second spectral range). In such cases, the first signaling protein receptor and the second signaling protein receptor are different protein receptors, each capable of activating a downstream signaling pathway important or necessary for growing and/or maintaining stem cells. Furthermore, the first light-activatable domain and the second light-activatable domain are different, and activatable by light at different wavelengths or within different spectral ranges. Such scenarios allow for the precise control of signaling pathway activation, and allow the user to modulate one signaling pathway without affecting the other. In a non-limiting example, the first signaling protein receptor is FGFR wherein, upon illumination of light at a first wavelength or within a first spectral range, the FGFR dimerizes or oligomerizes thereby activating a first signaling pathway (e.g., an ERK signaling pathway); and the second signaling protein receptor is TGFβR wherein, upon illumination of light at a second, different wavelength, or within a second, different spectral range, the TGFβR dimerizes or oligomerizes thereby activating a second signaling pathway (e.g., SMAD2/3 signaling pathway).

The stem cells used with the systems provided herein may be any desired stem cell. In some instances, the stem cells are pluripotent stem cells. In some instances, the stem cells are multipotent stem cells. In some cases, the stem cells are embryonic stem cells. In some cases, the stem cells are mesenchymal stem cells. In some cases, the stem cells are satellite cells or muscle stem cells. In some cases, the stem cells are fat stem cells. In certain embodiments, the stem cells described herein are mammalian stem cells. In some cases, the mammalian stem cells are selected from the group consisting of: human stem cells, bovine (cow) stem cells, ovine (sheep) stem cells, and porcine (pig) stem cells. In one example, the mammalian stem cells are bovine cells. The bovine cells may be, in some cases, from Wagyu bull or Angus bull. In some cases, the stem cells are avian stem cells, such as, but not limited to, chicken stem cells. In some cases, the stem cells are fish stem cells, such as, but not limited to, tuna stem cells.

Advantageously, the systems herein provide for growing and/or maintaining stem cells in suspension (rather than as, e.g., two-dimensional, adherent cell cultures). In some cases, the systems provided herein do not require the use of a feeder layer of cells. In some cases, the systems provided herein do not require the use of extracellular matrix components. In some cases, the systems involve growing or maintaining stem cells in a bioreactor. In some cases, the systems provided herein may involve the use of microcarriers (e.g., beads). In some cases, microcarriers may be coated with extracellular matrix components (e.g., to promote attachment of stem cells thereto).

In various aspects, the suspension culture has a volume of at least about 100 milliliters (mL). For example, the suspension culture may have a volume of at least about 150 mL, at least about 200 mL, at least about 250 mL, at least about 300 mL, at least about 350 mL, at least about 400 mL, at least about 450 mL, at least about 500 mL, at least about 550 mL, at least about 600 mL, at least about 650 mL, at least about 700 mL, at least about 750 mL, at least about 800 mL, at least about 850 mL, at least about 900 mL, at least about 950 mL, at least about 1000 mL, at least about 2000 mL, at least about 3000 mL, at least about 4000 mL, at least about 5000 mL, or greater. In some cases, the suspension culture has a volume of less than about 1000 mL. In some cases, the suspension culture has a volume of greater than about 1000 mL.

Any suitable cell culture media for growing and/or maintaining stem cells may be used. In some embodiments, the cell culture media comprises any of the following in an appropriate combination: isotonic saline, buffer, amino acids, sugars (e.g., glucose), serum or serum replacement, and other exogenously added factors. In some embodiments, the cell culture media comprises DMEM, F12, aMEM, Hepatostim™, RPMI, or combinations thereof, either in the presence or absence or serum. Suitable sera include calf serum, fetal calf serum, horse serum, or the like. In some embodiments, a serum supplement is used.

In some embodiments, the cell culture media is deficient in one or more factor required for growing and/or maintaining the stem cells. In some embodiments, the cell culture media is deficient in FGF, TGFβ, or both. In some embodiments, the cell culture media is deficient in FGF2, TGFβ1, activin, Nodal, LIF, or a combination thereof.

In various aspects, the systems described herein allow for the stem cells to be maintained in a pluripotent state for an extended period of time. In some embodiments, the stem cells are capable of being maintained in a pluripotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are capable of being maintained in a pluripotent state for at least 7 days. In some cases, the stem cells are capable of being maintained in a pluripotent state for at least 1 month. In some embodiments, the stem cells are maintained in a pluripotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are maintained in a pluripotent state for at least 7 days. In some cases, the stem cells are maintained in a pluripotent state for at least 1 month. In various aspects, the stem cells, after performing the methods provided herein, express one or more markers of pluripotency (e.g., OCT4).

In various aspects, the systems described herein allow for the stem cells to be maintained in a multipotent state for an extended period of time. In some embodiments, the stem cells are capable of being maintained in a multipotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are capable of being maintained in a multipotent state for at least 7 days. In some cases, the stem cells are capable of being maintained in a multipotent state for at least 1 month. In some embodiments, the stem cells are maintained in a multipotent state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are maintained in a multipotent state for at least 7 days. In some cases, the stem cells are maintained in a multipotent state for at least 1 month. In various aspects, the stem cells, after performing the methods provided herein, express one or more markers of multipotency.

In various aspects, the stem cells are capable of being maintained in an undifferentiated state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are capable of being maintained in an undifferentiated state for at least 7 days. In some cases, the stem cells are capable of being maintained in an undifferentiated state for at least 1 month. In various aspects, the stem cells are maintained in an undifferentiated state for at least about 1 week, at least about 2 weeks, at least about 3 weeks, at least about 4 weeks, at least about 1 month, at least about 2 months, at least about 3 months, at least about 4 months, at least about 5 months, at least about 6 months, or more than 6 months. In some cases, the stem cells are maintained in an undifferentiated state for at least 7 days. In some cases, the stem cells are maintained in an undifferentiated state for at least 1 month. In various aspects, the stem cells, after performing the methods provided herein, do not express one or more markers of differentiation. In various aspects, the stem cells do not express one or more cell or tissue-type specific markers. In various aspects, the stem cells, after performing the methods provided herein, express one or more markers of stemness. In various aspects, the stem cells, after performing the methods provided herein, express one or more markers of an undifferentiated state.

In various aspects, the systems provided herein are used to promote the growth of stem cells in suspension. In some cases, promoting the growth of stem cells comprises increasing proliferation rates. In some cases, promoting the growth of stem cells comprises maintaining proliferation rates.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993).

The section headings used herein are for organizational purposes only and are not to be construed as limiting the subject matter described.

### Numbered Embodiments

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed.
Embodiment 1: A method of growing and/or maintaining stem cells in suspension, the method comprising: (a) culturing the stem cells in a culture media in suspension, wherein the stem cells have been genetically engineered to express a first fusion protein, the first fusion protein comprising a first signaling protein receptor or a functional portion thereof and a first light-activatable domain; (b) exposing the stem cells to light thereby activating the first light-activatable domain resulting in activation of a downstream signaling pathway of the first signaling protein receptor, such that the stem cells are grown and/or maintained in suspension.
Embodiment 2: The method of embodiment 1, wherein the signaling protein receptor or functional portion thereof is a growth factor receptor.
Embodiment 3: The method of embodiment 1 or 2, wherein the culture media is deficient in one or more factor required for growing and/or maintaining the stem cells.
Embodiment 4: The method of embodiment 3, wherein the one or more factor is a ligand for the first signaling protein receptor or functional portion thereof.
Embodiment 5: The method of embodiment 4, wherein the one or more factor comprises one or more growth factor.
Embodiment 6: The method of embodiment 5, wherein the one or more growth factor is transforming growth factor beta (TGFβ).
Embodiment 7: The method of embodiment 5 or 6, wherein the one or more growth factor is a fibroblast growth factor (FGF), for example, FGF2.
Embodiment 8: The method of any one of embodiments 1-7, wherein the first signaling protein receptor or functional portion thereof is a fibroblast growth factor receptor (FGFR) or a transforming growth factor receptor (TGFR).
Embodiment 9: The method of any one of embodiments 1-8, wherein the first signaling protein receptor or functional portion thereof is selected from the group consisting of TGFβR1, TGFβR2, FGFR1, FGFR2, and any combination thereof.
Embodiment 10: The method of any one of embodiments 1-9, wherein the stem cells are mammalian stem cells.
Embodiment 11: The method of any one of embodiments 1-10, wherein the stem cells are human or bovine.
Embodiment 12: The method of any one of embodiments 1-11, wherein the stem cells are pluripotent stem cells or multipotent stem cells.
Embodiment 13: The method of embodiment 12, wherein the pluripotent stem cells are maintained, or are capable of being maintained, in a pluripotent state for at least 7 days.
Embodiment 14: The method of embodiment 12, wherein the pluripotent stem cells are maintained, or are capable of being maintained, in a pluripotent state for at least 1 month.
Embodiment 15: The method of embodiment 12, wherein the multipotent stem cells are maintained, or are capable of being maintained, in a multipotent state for at least 7 days.
Embodiment 16: The method of embodiment 12, wherein the multipotent stem cells are maintained, or are capable of being maintained, in a multipotent state for at least 1 month.
Embodiment 17: The method of any one of embodiments 1-16, wherein the stem cells are grown and/or maintained, or are capable of being grown and/or maintained, in suspension for at least 7 days.
Embodiment 18: The method of any one of embodiments 1-17, wherein the stem cells are grown and/or maintained, or are capable of being grown and/or maintained, in suspension for at least 1 month.
Embodiment 19: The method of any one of embodiments 1-18, wherein the stem cells remain, or are capable of remaining, in an undifferentiated state for at least 7 days.
Embodiment 20: The method of any one of embodiments 1-19, wherein the stem cells remain, or are capable of remaining, in an undifferentiated state for at least 1 month.
Embodiment 21: The method of any one of embodiments 1-20, wherein the stem cells are embryonic stem cells.
Embodiment 22: The method of any one of embodiments 1-21, wherein the stem cells are mesenchymal stem cells.
Embodiment 23: The method of any one of embodiments 1-22, wherein the stem cells are satellite cells or muscle stem cells.
Embodiment 24: The method of any one of embodiments 1-23, wherein the stem cells are fat stem cells.
Embodiment 25: The method of any one of embodiments 1-24, wherein the suspension culture has a volume of at least 100 milliliters (mL).
Embodiment 26: The method of any one of embodiments 1-25, wherein the suspension culture is contained within a bioreactor vessel.
Embodiment 27: The method of embodiment 26, wherein the bioreactor vessel has a total volume of at least 100 milliliters (mL).
Embodiment 28: The method of any one of embodiments 1-27, wherein the light-activatable domain is selected from the group consisting of: a Light-Oxygen-Voltage (LOV) photoreceptor domain, a LOV2 photoreceptor domain, a Cryptochrome (CRY) photoreceptor domain, Blue-light-using FAD (BLUF) photoreceptor domain, a Phytochrome (PHY) photoreceptor domain, a CIBN (N-terminal domain of CIB1 (cryptochrome-interacting basic-helix-loop-helix protein 1)) domain, a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof.
Embodiment 29: The method of any one of embodiments 1-28, wherein the exposing of (b) comprises exposing the stem cells to light having one or more illumination parameters.
Embodiment 30: The method of embodiment 29, wherein the one or more illumination parameters comprises light intensity.
Embodiment 31: The method of embodiment 29, wherein the one or more illumination parameters comprises a temporal pattern of illumination.
Embodiment 32: The method of embodiment 31, wherein the temporal pattern comprises a light stimulus duration of at least about 5 minutes.
Embodiment 33: The method of embodiment 31 or 32, wherein the temporal pattern comprises an interstimulus duration of from about 20 minutes to about 250 minutes.
Embodiment 34: The method of any one of embodiments 1-33, wherein the first fusion protein, after the exposing to light of (b), dimerizes or oligomerizes.
Embodiment 35: The method of any one of embodiments 1-34, wherein the stem cells are genetically engineered to express a second fusion protein comprising a second signaling protein receptor or functional portion thereof and a second light-activatable domain.
Embodiment 36: The method of embodiment 35, wherein the second signaling protein receptor or functional portion thereof is different from the first signaling protein receptor or functional portion thereof.
Embodiment 37: The method of embodiment 35 or 36, wherein the second light-activatable domain is different from the first light-activatable domain.
Embodiment 38: The method of any one of embodiments 35-37, wherein the first signaling protein receptor or functional portion thereof is TFGβR1 or TGFβR2, and the second signaling protein receptor or functional portion thereof is FGFR1 or FGFR2.
Embodiment 39: The method of any one of embodiments 1-38, wherein the downstream signaling pathway is a SMAD2/3 signaling pathway.
Embodiment 40: The method of any one of embodiments 1-39, wherein the downstream signaling pathway is an ERK signaling pathway.
Embodiment 41: The method of any one of embodiments 1-40, wherein the first signaling protein receptor or functional portion thereof, the second signaling protein receptor or functional portion thereof, or both, does not comprise a fibroblast growth factor receptor (FGFR).
Embodiment 42: The method of any one of embodiments 1-41, wherein the exposing of (b) comprises exposing the stem cells to light at a wavelength from 100 nm to 1 mm.
Embodiment 43: The method of any one of embodiments 1-42, wherein the exposing of (b) comprises exposing the stem cells to ultraviolet light, visible light, near infrared light, infrared light, or a combination thereof.
Embodiment 44: The method of embodiment 43, wherein visible light is blue light, red light, green light, or a combination thereof.
Embodiment 45: The method of any one of embodiments 1-44, wherein, at 7 days or greater in suspension culture, the stem cells express one or more of markers of stemness.
Embodiment 46: A system for growing and/or maintaining stem cells, the system comprising: (a) a bioreactor vessel comprising a culture media; (b) a plurality of stem cells in suspension in the culture media, wherein the stem cells have been genetically engineered to express a first fusion protein comprising a first signaling protein receptor or functional portion thereof and a first light-activatable domain; and (c) one or more light source for exposing the stem cells to light to activate the first light-activatable domain resulting in a downstream signaling pathway of the first signaling protein receptor or functional portion thereof, such that the stem cells are grown and/or maintained in suspension.
Embodiment 47: The system of embodiment 46, wherein the bioreactor vessel has a total volume of at least 100 milliliters (mL).
Embodiment 48: The system of embodiment 46 or 47, wherein the one or more light source comprises one or more light emitting diodes (LEDs).
Embodiment 49: The system of embodiment 48, wherein the one or more LEDs comprises at least two different LEDs.
Embodiment 50: The system of embodiment 49, wherein the at least two different LEDs emit light at different wavelengths.
Embodiment 51: The system of embodiment 46 or 47, wherein the one or more light source comprises one or more lasers.
Embodiment 52: The system of embodiment 46 or 47, wherein the one or more light source comprises an incandescent light source.
Embodiment 53: The system of any one of embodiments 46-52, wherein the one or more light source is located inside the bioreactor vessel, or located on an interior surface of the bioreactor vessel.
Embodiment 54: The system of any one of embodiments 46-52, wherein the one or more light source is located outside the bioreactor vessel, or on an exterior surface of the bioreactor vessel.
Embodiment 55: The system of embodiment 54, wherein the bioreactor vessel comprises at least one wall or surface that is optically transparent.
Embodiment 56: The system of any one of embodiments 46-55, further comprising a temperature source for controlling a temperature of the culture media.
Embodiment 57: The system of any one of embodiments 46-56, further comprising an agitation source for agitating the culture media.
Embodiment 58: The system of any one of embodiments 46-57, wherein the system is configured to provide light from the one or more light source in a pattern.
Embodiment 59: The system of embodiment 58, wherein the pattern is a spatial pattern, a temporal pattern, or both.
Embodiment 60: The system of embodiment 59, wherein the temporal pattern comprises a light stimulus duration and an interstimulus duration.
Embodiment 61: The system of embodiment 60, wherein the light stimulus duration is at least about 5 minutes.
Embodiment 62: The system of embodiment 60 or 61, wherein the interstimulus duration is from about 20 minutes to about 250 minutes.
Embodiment 63: The system of any one of embodiments 46-62, wherein the first signaling protein receptor is a growth factor receptor.
Embodiment 64: The system of any one of embodiments 46-63, wherein the cell culture media is deficient in one or more factor required for growing and/or maintaining the stem cells.
Embodiment 65: The system of embodiment 64, wherein the one or more factor is a ligand for the first signaling protein receptor or functional portion thereof.
Embodiment 66: The system of embodiment 65, wherein the one or more factor comprises one or more growth factor.
Embodiment 67: The system of embodiment 66, wherein the one or more growth factor is transforming growth factor beta (TGFβ).
Embodiment 68: The system of embodiment 66 or 67, wherein the one or more growth factor is a fibroblast growth factor (FGF), for example, FGF2.
Embodiment 69: The system of any one of embodiments 46-68, wherein the first signaling protein receptor or functional portion thereof is a fibroblast growth factor receptor (FGFR) or a transforming growth factor receptor (TGFR).
Embodiment 70: The system of any one of embodiments 46-69, wherein the first signaling protein receptor or functional portion thereof is selected from the group consisting of: TGFβR1, TGFβR2, FGFR1, FGFR2, and any combination thereof.
Embodiment 71: The system of any one of embodiments 46-70, wherein the stem cells are mammalian stem cells.
Embodiment 72: The system of any one of embodiments 46-71, wherein the stem cells are human or bovine.
Embodiment 73: The system of any one of embodiments 46-72, wherein the stem cells are pluripotent stem cells or multipotent stem cells.
Embodiment 74: The system of embodiment 73, wherein the pluripotent stem cells are maintained, or are capable of being maintained, in a pluripotent state for at least 7 days.
Embodiment 75: The system of embodiment 73, wherein the pluripotent stem cells are maintained, or are capable of being maintained, in a pluripotent state for at least 1 month.
Embodiment 76: The system of embodiment 73, wherein the multipotent stem cells are maintained, or are capable of being maintained, in a multipotent state for at least 7 days.
Embodiment 77: The system of embodiment 73, wherein the multipotent stem cells are maintained, or are capable of being maintained, in a multipotent state for at least 1 month.
Embodiment 78: The system of any one of embodiments 46-77, wherein the stem cells are grown and/or maintained, or are capable of being grown and/or maintained, in suspension for at least 7 days.
Embodiment 79: The system of any one of embodiments 46-78, wherein the stem cells are grown and/or maintained, or are capable of being grown and/or maintained, in suspension for at least 1 month.
Embodiment 80: The system of any one of embodiments 46-79, wherein the stem cells remain, or are capable of remaining, in an undifferentiated state for at least 7 days.
Embodiment 81: The system of any one of embodiments 46-80, wherein the stem cells remain, or are capable of remaining, in an undifferentiated state for at least 1 month.
Embodiment 82: The system of any one of embodiments 46-81, wherein the stem cells are embryonic stem cells.
Embodiment 83: The system of any one of embodiments 46-81, wherein the stem cells are mesenchymal stem cells.
Embodiment 84: The system of any one of embodiments 46-81, wherein the stem cells are satellite cells or muscle stem cells.
Embodiment 85: The system of any one of embodiments 46-81, wherein the stem cells are fat stem cells.
Embodiment 86: The system of any one of embodiments 46-85, wherein the culture media has a volume of at least 100 milliliters (mL).
Embodiment 87: The system of any one of embodiments 46-86, wherein the light-activatable domain is selected from the group consisting of: a Light-Oxygen-Voltage (LOV) photoreceptor domain, a LOV2 photoreceptor domain, a Cryptochrome (CRY) photoreceptor domain, Blue-light-using FAD (BLUF) photoreceptor domain, a Phytochrome (PHY) photoreceptor domain, a CIBN (N-terminal domain of CIB1 (cryptochrome-interacting basic-helix-loop-helix protein 1)) domain, a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof.
Embodiment 88: The system of any one of embodiments 46-87, wherein the first fusion protein, after the exposing to light of (b), dimerizes or oligomerizes.
Embodiment 89: The system of any one of embodiments 46-88, wherein the stem cells are genetically engineered to express a second fusion protein comprising a second signaling protein receptor or functional portion thereof and a second light-activatable domain.
Embodiment 90: The system of embodiment 89, wherein the second signaling protein receptor or functional portion thereof is different from the first signaling protein receptor or functional portion thereof.
Embodiment 91: The system of embodiment 89 or 90, wherein the second light-activatable domain is different from the first light-activatable domain.
Embodiment 92: The system of any one of embodiments 89-91, wherein the first signaling protein receptor or functional portion thereof is TFGbR1 or TGFβR2, and the second signaling protein receptor or functional portion thereof is FGFR1 or FGFR2.
Embodiment 93: The system of any one of embodiments 46-92, wherein the downstream signaling pathway is a SMAD2/3 signaling pathway.
Embodiment 94: The system of any one of embodiments 46-92, wherein the downstream signaling pathway is an ERK signaling pathway.
Embodiment 95: The system of any one of embodiments 46-94, wherein the first signaling protein receptor or functional portion thereof, the second signaling protein receptor or functional portion thereof, or both, does not comprise a fibroblast growth factor receptor (FGFR).
Embodiment 96: The system of any one of embodiments 46-95, wherein the exposing of (b) comprises exposing the stem cells to light at a wavelength from 100 nm to 1 mm.
Embodiment 97: The system of any one of embodiments 46-96, wherein the exposing of (b) comprises exposing the stem cells to ultraviolet light, visible light, near infrared light, infrared light, or a combination thereof.
Embodiment 98: The system of embodiment 97, wherein visible light is blue light, red light, green light, or a combination thereof.

### EXAMPLES

### Example 1. Engineering of Stem Cells to Express a Fusion Protein Containing a Light-Activatable Domain and a Signaling Protein Receptor

Adipose stem cells were extracted from fresh adipose tissue samples of adult Wagyu bull. Briefly, adipose tissue was minced with a sterile scalpel, added to 25 ml digestion solution (2 mg/ml Collagenase II (Sigma, C2-BIOC), 1% penicillin/streptomycin/amphotericin B (Lonza, 17-745E), 10 μM ROCK inhibitor Y-27632 (Tocris,1 254)), leading to a total volume of 40 ml in a 50 ml centrifuge tube, and incubated at 37° C. for 60 minutes with inversion every 2 minutes. The tube was centrifuged at 300×g for 5 minutes, resulting in a pellet at the bottom and a plug of fat at the top, which was discarded along with the supernatant. The pellet was washed with 25 ml growth medium (DMEM (Sigma, SLM-021) with 10% fetal bovine serum (Avantor, 89510-186) and 1% penicillin/streptomycin/amphotericin B, then resuspended in 10 ml growth medium and transferred to a T75 tissue culture flask. Cells were incubated undisturbed at 37° C. and 5% CO₂ for 24 hours, and thereafter were expanded for multiple passages with changes of growth medium every 2 days.

Myogenic stem cells were extracted from fresh biceps femoris samples of an adult Angus bull. Briefly, 2.5 grams of muscle tissue were minced with dissection scissors then incubated in 7000 U Collagenase II for 1 hour at 37° C. Collagenase solution was then removed and replaced with 1000 U Collagenase II and 11 U Dispase (Corning, 354235). Samples were incubated in digestive enzymes for another 30 minutes at 37° C. After 30 minutes of enzymatic digestion, samples were mechanically digested by repeated trituration with a 20 gauge needle. Enzymes were then removed, and samples were filtered by sequentially passing through 40 μm and 35 μm cell strainers. Samples were then resuspended in 10 mL of wash media (Ham's F10 (Cytiva, SH30025.01), 10% horse serum (Cytiva, SH30074.04IR2540), 1% penicillin/streptomycin/amphotericin B), and muscle stem cells were further enriched from bulk tissue prep by pre-plating. Briefly, 10 mL of bulk tissue isolate were deposited onto an uncoated 10 cm polystyrene dish and incubated at 37° C. and 5% CO₂ for 30 minutes. After 30 minutes, all non-adherent cells in the supernatant were removed and redeposited into a new dish, leaving behind the rapid adhering, non-stem cell fraction. This was repeated for a total of four pre-plating steps. Finally, the slow adhering fraction, enriched for muscle resident stem cells, was deposited onto a collagen (Sigma, C8919-20ML) coated 10 cm dish and incubated overnight at 37° C. and 5% CO₂. The following day, wash media was swapped to growth media (DMEM, 10% fetal bovine serum, 1% penicillin/streptomycin/amphotericin B, 2.5 ng/mL FGFb (Fisher Scientific, 2099-FB-025), and cells were expanded for multiple passages.

Wagyu adipose stem cells and Angus myogenic stem cells were engineered to express a fusion protein comprising a light-activatable domain fused to a signaling receptor protein. A plasmid containing a nucleic acid sequence encoding the cytoplasmic domain of bovine fibroblast growth factor receptor 1 (FGFR1) (residues 463 to 886 of Uniprot A0A3Q1LUE0) fused to a LOV domain from Vaucheria frigida Aureochrome1 (residues 204-348 of Uniprot A8QW55) was constructed under the control of a constitutive promoter. The LOV domain homodimerizes in response to blue light; when fused to the cytoplasmic domain of FGFR1, FGFR signaling is activated upon illumination with blue light. The FGFR-LOV protein was also fused to a FLAG epitope allowing for detection by anti-FLAG antibodies. The plasmid also contained a puromycin resistance gene and a membrane-localized red fluorescent protein, both for selection. The entire nucleotide sequence comprising these elements was flanked by the 5′ and 3′ ITR sequences of the piggyBac transposon. The insert sequence of each plasmid from the 5′ ITR to the 3′ ITR, inclusive, was cloned into the pUC57-Kan backbone (GenScript).

Plasmids were prepared with <0.01 EU/μg endotoxin. Insert cassettes from the pFGFR-LOV plasmid were integrated into the genome using the piggyBac transposon system. Nucleofection was performed using the 4D-Nucleofector X Unit (Lonza). A total of 140,000 cells, 1 μg of donor plasmid, and 0.2 μg of piggyBac transposase helper plasmid were resuspended in 20 μL P2 Primary Cell Nucleofector Solution with Supplement 1 (Lonza) before being transferred to a well in the Nucleocuvette Strip. Each condition was performed in duplicate in two wells of the Nucleocuvette Strip using the program EN150. The cells were then gently resuspended in 1 mL of pre-warmed growth medium, transferred to a 6-well plate, and grown overnight at 37° C. and 5% CO₂. The medium was refreshed 24 hours post-nucleofection. Puromycin (1.1 μg/ml) was included in the medium for selection of engineered cells beginning 48 hours post-nucleofection for 7 days. Stable integration of the cassette was confirmed by genotyping PCR and expression of the fusion protein by Western blot several weeks after transfection.

To expand clones from a single cell, cells were trypsinized for 5 minutes at 37° C. in the incubator to detach them from the surface. Cell clumps were dissociated into a single cell suspension by trituration for ten times. Dilution of the cells with growth medium (DMEM/F-12 (Cytiva, SH30023.FS) containing 10% fetal bovine serum (Avantor, 89510-186) was followed by centrifugation in order to remove trypsin and a suspension of 600 cells/ml was prepared in the growth medium. 100 μL (60 cells) was loaded into the inlet adaptor of a Smart Aliquotor CE (iBioChips). Cells were slowly pipetted through the Smart Aliquotor, and the inlet adaptor was removed after loading the cells. Each well in the Smart Aliquotor was examined immediately after cell isolation in order to identify the wells that contained single cells. 10 mL of pre-warmed growth medium was then added to the Smart Aliquotors before transferring them back to the incubator at 37° C. and 5% C02. Single cells in each well were monitored daily to track their growth. Expansion of single cell derived colonies to a 96-well plate was performed once they reached 50% confluency in the Smart Aliquotor, followed by further expansion in tissue culture flasks. Stable integration of the cassette was confirmed by genotyping PCR and expression of the protein by Western blot several weeks after transfection.

### Example 2. FGF Signaling is Activated by Light in Stem Cells Expressing FGFR-LOV Fusion Protein

This example demonstrates that stem cells engineered to express a fusion protein comprising a light-activatable domain fused to a signaling protein receptor (FGFR-LOV), as described in Example 1, activate FGF signaling in response to illumination with light, as measured by the phosphorylation of the downstream signaling component ERK1/2.

Briefly, 96 well glass bottomed plates (Cellvis, P96-1.5H-N) were coated with truncated vitronectin at 1.5 μg/cm² (ThermoFisher Scientific, A14700). Plates were incubated for 2 hours at room temperature and washed with phosphate buffered saline (PBS) (Cytiva, SH30256.FS). Wild-type cells and engineered cells were seeded at 3000 cells per well in media containing DMEM F-12 with glutamine (Cytiva, SH30271.FS), 10% fetal bovine serum (Avantor, 89510-186), and 1% penicillin/streptomycin/amphotericin B. The following day, cells were washed with PBS and serum-free media without FGF was added to all the wells, initiating a 24 hour FGF starvation. After 24 hours, 50 ng/ml FGF2 (Fisher Scientific, 2099-FB-025) was added to select wells. The plate was placed on the illuminator for 10 minutes of illumination of select wells at 470 nm wavelength, 5 μW/mm². The plate was then taken off the illuminator, media was removed, the wells were washed with PBS and cells were fixed with 4% paraformaldehyde (Boster Bio, AR1068) for 20 minutes at room temperature. Following fixation, cells were washed with PBS and permeabilized using a buffer containing PBS and 0.2% Triton-X100 (Sigma, 93418) for 10 minutes at room temperature. Cells were washed again with PBS and a blocking buffer containing PBS, 2% bovine serum albumin (Sigma, A6003-5G) and 0.1% Tween-20 (Sigma, P7949-100ML) was applied to the cells. The cells were kept on a plate rocker at room temperature for 1 hour. Anti-ERK1 (phospho T202)+ERK2 (phospho T185) antibody (abcam, ab21403), which specifically bind to the phosphorylated forms of ERK1/2, was diluted 1 in 400 in blocking buffer, added to the cells and kept at 4° C. overnight. The next day, the antibody solution was removed, washed with PBS containing 0.1% Tween-20. Goat anti-rabbit IgG Alexa Fluor 488 (ThermoFisher Scientific, A27034) was diluted 1 in 1000 in blocking buffer and placed on a plate shaker for 1 hour at room temperature. Cells were washed with PBS and Hoechst (ThermoFisher Scientific, 62249) was added at 10 μg/ml and placed on a plate rocker for 20 minutes at room temperature. Cells were washed with PBS and imaged on a Leica DMi8 Thunder fluorescence microscope.

Nuclear masks were made in the Hoechst channel by applying Thunder Instant Computational Clearing, auto-contrast, and gaussian blur with sigma 1.5, then using Otsu global thresholding and the fill-holes method to segment the cell nuclei. Objects were measured with the LasX software measurement module and outliers filtered based on size, roundness, and intensity. To measure phosphorylated ERK1/2 (pERK) intensity, Thunder Instant Computational Clearing was applied to the AlexaFluor488 channel. The segmented Hoechst channel was used as a binary mask and the mean intensity of the AlexaFluor488 in the nucleus of each cell measured using LasX. Values were plotted as a geometric density distribution using ggplot2 in python, grouped by well with each cell as a data point, to observe the distribution shift of the mean intensity of pERK in the nucleus for each population.

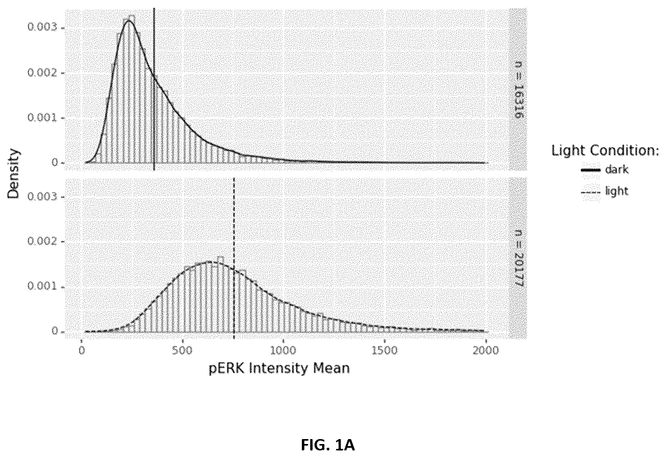

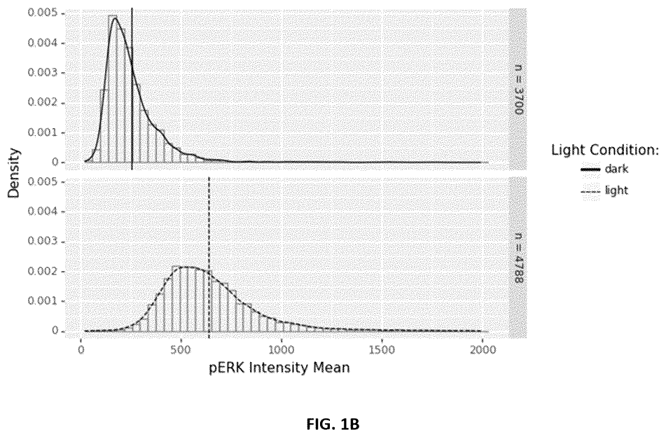

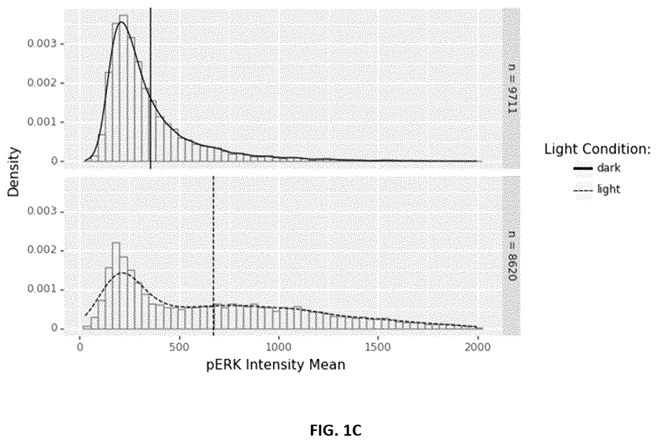

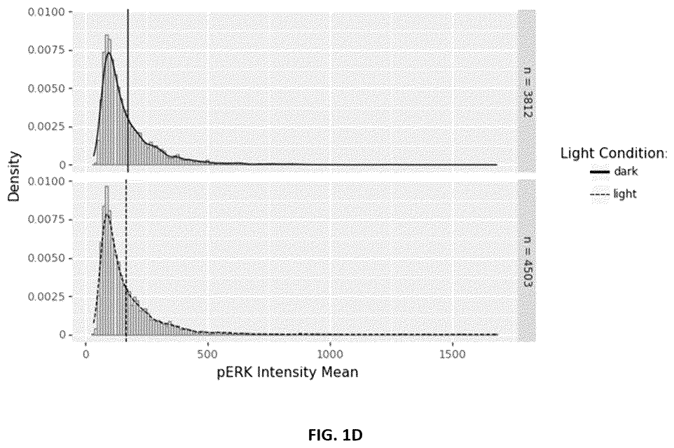

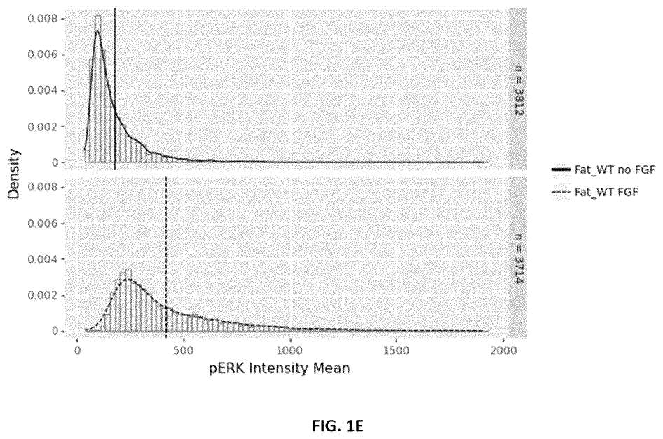

FIGS. 1A-1C demonstrate that, under some conditions, such as those described in these Examples, various stem cell populations engineered to express FGFR-LOV activated FGF signaling in response to blue light, including a mixed adipose stem cell population (FIG. 1A; “Fat_FGFR-LOV_mixed”), a clonal adipose stem cell population (FIG. 1B; “Fat_FGFR-LOV_clone”), and a muscle stem cell population (FIG. 1C; “Myo_FGFR-LOV”). In contrast, wild-type adipose stem cells (e.g., not expressing FGFR-LOV) did not activate FGF signaling in response to blue light (FIG. 1D). As a positive control, wild-type adipose stem cells (e.g., not expressing FGFR-LOV) activated FGF signaling in response to exogenous addition of FGF2 (FIG. 1E) demonstrating that the assay is capable of measuring FGF signaling.

### Example 3. Adipose Stem Cells Expressing a Light-Activatable Domain Fused to a Signaling Protein Receptor are Grown and Maintained in Suspension Culture in the Absence of Exogenous Growth Factors Upon Illumination with Light

This example demonstrates that adipose stem cells engineered to express a light-activatable domain fused to a signaling protein receptor can be grown and maintained in suspension culture with light, without the addition of exogenous growth factors.

Prior to inoculation in a bioreactor, stem cells were cultured in polystyrene tissue culture flasks in humidified incubators held at 37° C. and supplemented with 5% CO₂. Cells were cultured in DMEM/F-12 cell culture medium including L-glutamine and HEPES (Cytiva, SH30023.FS), and supplemented with 10% FBS, 1% penicillin/streptomycin/amphotericin B, and 2.5 mM Glutamax. Cells were passaged prior to reaching confluency, and were dissociated from flasks using TrypLE (ThermoFisher Scientific, 12604013).

A DASbox 250 mL bioreactor system (Eppendorf) was used for all suspension cell culture. For all experiments, BioBLU 0.3c single-use vessels (Eppendorf) were used. Cells were cultured on Cytodex 1 microcarriers (Cytiva) in the bioreactors. Microcarriers were prepared by aliquoting 833 mg dry microcarriers per bioreactor vessel. Microcarrier aliquots were hydrated in centrifuge tubes with PBS and sterilized in an autoclave. PBS was removed and cells were resuspended in serum-free cell culture media. Microcarriers and media were added to bioreactor vessels until each vessel contained 833 mg (dry weight) microcarriers and 90 mL serum-free media. Vessels were then placed in DASbox units and the system was set to the following setpoints: 37° C., pH 7.3, 60 rpm counterclockwise agitation, 3.0 sL/hr gassing with overlay air and sparged CO₂. Vessels were allowed to equilibrate to these setpoints.

Cells were dissociated from tissue culture flasks using TrypLE and diluted to 7.5×10⁵ cells/mL in serum-free media. 10 mL of this cell suspension was added to each bioreactor vessel for a total of 7.5×10⁶ cells per bioreactor, bringing the volume to 100 mL per bioreactor. After inoculation, vessels were agitated for 30 seconds every 30 minutes for the next 4 hours. Then, agitation was stopped overnight to allow cells to attach to microcarriers. The following day, 150 mL of fresh media was added to each bioreactor to bring the total working volume to 250 mL. On day 5, 80% (200 mL) of the media in each vessel was exchanged with fresh media.

3 mL samples were collected daily for quantification using a syringe connected to the sampling line of the BioBLU vessel. Total cells per mL were quantified by following the protocol described in Chemometec Technical note No. 0221 Rev. 1.2. Briefly, cells attached to microcarriers were lysed to release nuclei. The nuclei were then stabilized to prevent lysis. The nuclei were drawn into a Vial-Cassette, within which they were stained with Hoechst. The nuclei were then counted on the NC-200 cell counter (Chemometec). Blue light (approximately 470 nm) was delivered through the transparent bioreactor wall with intensities and timings as described in Table 2 below.

TABLE 2
Illumination Parameters

| | Avg light | Light intensity | Intensity | | | |
|---|---|---|---|---|---|---|
| | intensity | (at vessel | (at vessel | Stimulus | Interstimulus | |
| | in vessel | wall) | center) | duration | interval | FGF2 |
| Condition ID | [μW/mm²] | [μW/mm²] | [μW/mm²] | [min] | [min] | [ng/mL] |
| Fat_WT − FGF dark | 0 | 0 | 0 | 0 | 0 | 0 |
| Fat_WT + FGF dark | 0 | 0 | 0 | 0 | 0 | 5 |
| Fat_FGFR − | 1 | 4 | 0.1 | 5 | 115 | 0 |
| LOV_mixed light1 | | | | | | |
| Fat_FGFR − | 5 | 20 | 0.5 | 5 | 115 | 0 |
| LOV_clone light2 | | | | | | |

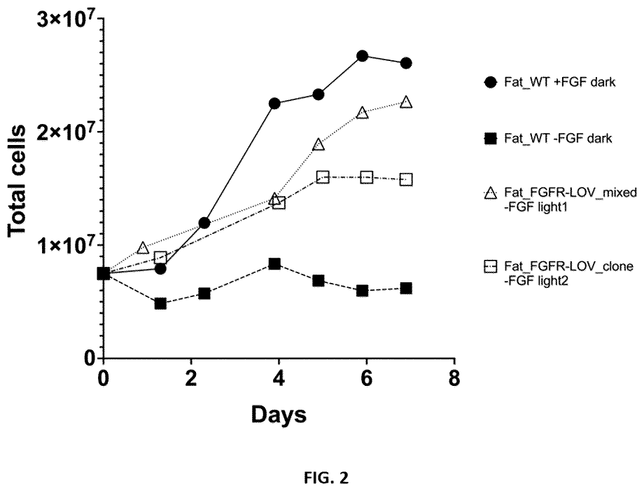

FIG. 2 demonstrates that, under some conditions, such as those described in these Examples, stem cells engineered to express FGFR-LOV, when exposed to blue light, proliferated for at least 7 days in suspension in the absence of exogenous FGF. FIG. 2 demonstrates that both a mixed population of adipose stem cells expressing FGFR-LOV (“Fat_FGFR-LOV_mixed−FGF light1”) and a clonal population of adipose stem cells expressing FGFR-LOV (“Fat_FGFR-LOV_clone−FGF light2”) proliferated for at least 7 days in suspension in the absence of exogenous FGF upon illumination with blue light with different illumination parameters. Wild-type adipose stem cells (e.g., not expressing FGFR-LOV) also proliferated for at least 7 days in suspension when exposed to exogenous FGF (“Fat_WT+FGF dark”). In contrast, wild-type adipose stem cells (e.g., not expressing FGFR-LOV) not exposed to exogenous FGF did not proliferate (“Fat_WT−FGF dark”). This data demonstrates that stem cells expressing a fusion protein comprising a light-activatable domain fused to a signaling protein receptor (e.g., FGFR-LOV) are capable of being grown in suspension in response to light under various illumination parameters without the need to add exogenous growth factors (e.g., FGF).

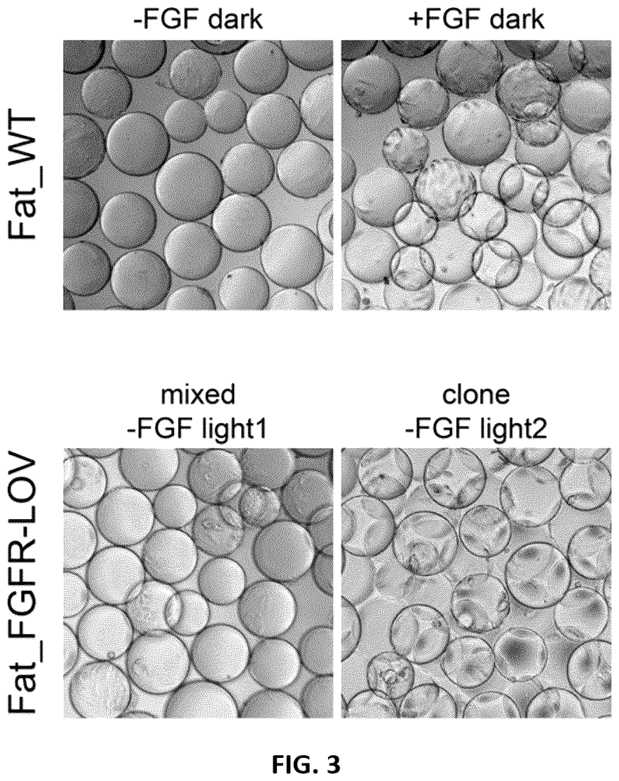

To further demonstrate that stem cells expressing FGFR-LOV proliferate in suspension in response to light, images of the microcarriers were taken. 50 μl of microcarrier suspension was withdrawn from the bioreactor on day 7 of culture, transferred to a well of a 96-well glass bottomed plate (Cellvis, P96-1.5H-N), and the microcarriers were allowed to settle to the bottom of the well. Microcarriers were imaged with a Leica DMi8 Thunder microscope using brightfield illumination and a 5× objective. FIG. 3 demonstrates that adipose stem cells engineered to express FGFR-LOV proliferated for at least 7 days in suspension in response to light in the absence of exogenous FGF (“Fat_FGFR-LOV_mixed−FGF light1” and “Fat_FGFR-LOV_clone−FGF light2”). The data further demonstrates that positive control wild-type adipose stem cells exposed to exogenous FGF also proliferated for at least 7 days in suspension (“Fat_WT+FGF dark), whereas wild-type adipose stem cells not exposed to exogenous FGF did not proliferate (“Fat_WT−FGF dark”).

Maintenance of stemness was also assessed in these cell lines. After 7 days of growth in suspension, cells were detached from microcarriers using trypsin, plated at a density of 15,000 cells/cm², and incubated at 37° C. and 5% CO₂ overnight in growth media (DMEM-F12 containing 10% FBS). The following day, cells were fixed for 20 minutes in cold 4% PFA, permeabilized using 0.2% Triton X-100, and blocked in 1% BSA, 10% FBS. After blocking, cells were stained overnight at 4° C. for CD29 (Biolegend 303007, 5 μg/mL), a cell surface antigen enriched on adipose-derived stem cells (PMID: 19995482, 27133085, 28582278). Primary antibodies were then washed off, and cells were stained with a donkey anti-mouse 555 antibody (ThermoFisher Scientific, A32773, 1 μg/mL) and Hoechst 33342 for 1 hour at room temperature. After 1 hour, secondary antibodies were washed off, and cells were imaged using a Leica DMi8 Thunder fluorescence microscope.

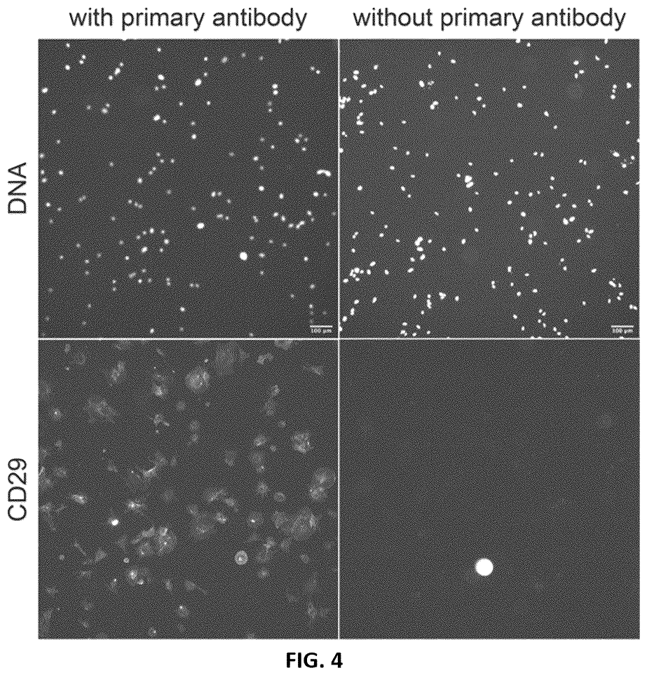

FIG. 4 shows that the clonal population of adipose stem cells expressing FGFR-LOV (“Fat_FGFR-LOV_clone”) expressed the adipose stem cell marker CD29 after 7 days in suspension culture with light in the absence of exogenous FGF. This data demonstrates that these cells expressed at least one cell surface marker enriched on adipose stem cells, suggesting that sternness was maintained after 7 days in suspension culture with light.

### Example 4. Muscle Stem Cells Expressing a Light-Activatable Domain Fused to a Signaling Protein Receptor can be Grown and Maintained in Suspension Culture in the Absence of Exogenous Growth Factors Upon Illumination with Light

This example demonstrates that muscle stem cells engineered to express a light-activatable domain fused to a signaling protein receptor can be grown and maintained in suspension culture with light, without the addition of exogenous growth factors.

Prior to inoculation in a bioreactor, cells were cultured in collagen-coated polystyrene tissue culture flasks in humidified incubators held at 37° C. and supplemented with 5% CO₂. Cells were cultured in high-glucose DMEM cell culture medium including L-glutamine and HEPES (Cytiva, SH30023.FS), and supplemented with 10% FBS, 1% penicillin/streptomycin/amphotericin B, and 2.5 ng/mL FGF2. Cells were passaged prior to reaching confluency, and were dissociated from flasks using 0.25% trypsin.

A DASbox 250 mL bioreactor system (Eppendorf) was used for all suspension cell culture. For all experiments, BioBLU 0.3c single-use vessels (Eppendorf) were used. Cells were cultured on Cytodex 1 microcarriers (Cytiva) in the bioreactors. Microcarriers were prepared by aliquoting 833 mg dry microcarriers per bioreactor vessel. Microcarrier aliquots were hydrated in centrifuge tubes with PBS and sterilized in an autoclave. PBS was removed and cells were resuspended in serum-free cell culture media. Microcarriers and media were added to bioreactor vessels until each vessel contained 833 mg (dry weight) microcarriers and 90 mL serum-free media. Vessels were then placed in DASbox units and the system was set to the following setpoints: 37° C., pH 7.3, 40 rpm counterclockwise agitation, 3.0 sL/hr gassing with overlay air and sparged CO₂. Vessels were allowed to equilibrate to these setpoints.

Cells were dissociated from tissue culture flasks using 0.25% trypsin. Wild-type cells were diluted to 4.82×10⁵ cells/mL in serum-free media. 14 mL of this cell suspension was added to each bioreactor vessel for a total of 6.75×10⁶ cells per bioreactor, bringing the volume to 104 mL per bioreactor. FGFR-LOV expressing cells were diluted to 7.5×10⁵ cells/mL in serum-free media. 10 mL of this cell suspension was added to each bioreactor vessel for a total of 7.5×10⁶ cells per bioreactor, bringing the volume to 100 mL per bioreactor. After inoculation, vessels were agitated for 30 seconds every 30 minutes for the next 4 hours. Then, agitation was stopped overnight to allow cells to attach to microcarriers. The following day, an appropriate volume of fresh media was added to each bioreactor to bring the total working volume to 250 mL per vessel. On day 5, 80% (200 mL) of the media in each vessel was exchanged with fresh media. Samples were collected and analyzed in the same manner as described in Example 3. Blue light (approximately 470 nm) was delivered through the transparent bioreactor wall with intensities and timings as described in Table 3 below.

TABLE 3
Illumination Parameters

| | Avg light | Light intensity | Intensity | | | |
|---|---|---|---|---|---|---|
| | intensity | (at vessel | (at vessel | Stimulus | Interstimulus | |
| | in vessel | wall) | center) | duration | interval | FGF2 |
| Condition ID | [μW/mm²] | [μW/mm²] | [μW/mm²] | [min] | [min] | [ng/mL] |
| Myo_WT − FGF dark | 0 | 0 | 0 | 0 | 0 | 0 |
| Myo_WT + FGF dark | 10 | 0 | 0 | 0 | 0 | 10 |
| Myo_FGFR − LOV | 5 | 20 | 0.5 | 5 | 25 | 0 |
| light1 | | | | | | |
| Myo_FGFR − LOV | 5 | 20 | 0.5 | 5 | 115 | 0 |
| light2 | | | | | | |
| Myo_FGFR − LOV | 5 | 20 | 0.5 | 5 | 235 | 0 |
| light3 | | | | | | |
| Myo_FGFR − LOV | 0.5 | 2 | 0.05 | 5 | 25 | 0 |
| light4 | | | | | | |
| Myo_FGFR − LOV | 0.5 | 2 | 0.05 | 5 | 115 | 0 |
| light5 | | | | | | |
| Myo_FGFR − LOV | 0.5 | 2 | 0.05 | 5 | 235 | 0 |
| light6 | | | | | | |

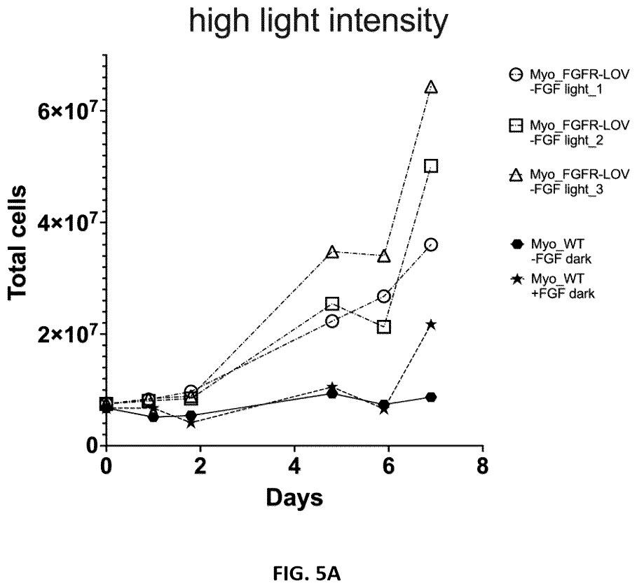

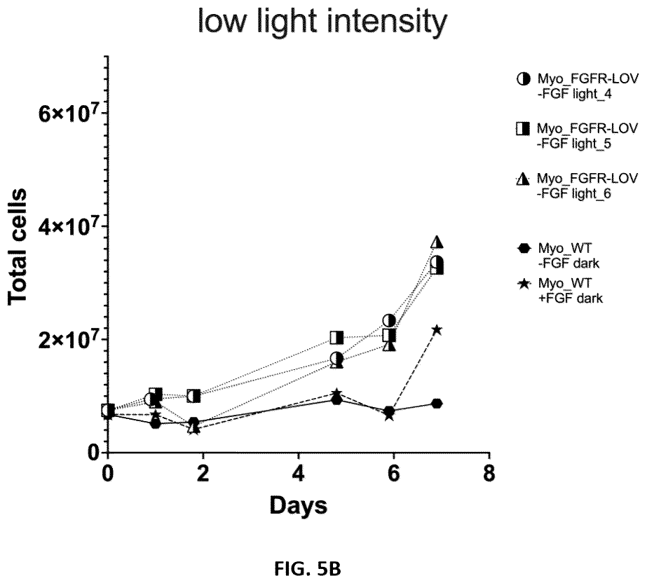

FIGS. 5A and 5B demonstrate that muscle stem cells engineered to express FGFR-LOV, when exposed to blue light under various illumination parameters, proliferated for at least 7 days in suspension in the absence of exogenous FGF. The results depicted in FIGS. 5A and 5B were from the same experiment and used the same control. FIG. 5A depicts results using illumination with light at high intensity, and FIG. 5B depicts results from the same experiment using illumination with light at low intensity. FIGS. 5A and 5B demonstrate that muscle stem cells engineered to express FGFR-LOV (“Myo_FGFR-LOV”) proliferated for at least 7 days in suspension in the absence of exogenous FGF upon illumination with blue light at high and low intensities (FIG. 5A: high intensity=average light intensity in vessel during illumination of 5 μW/mm²; FIG. 5B: low intensity=average light intensity in vessel during illumination of 0.5 μW/mm²) with various different light pulsing protocols (light1 and light4=5 minutes stimulus duration, 25 minute interstimulus duration; light2 and light5=5 minute stimulus duration, 115 minute interstimulus duration; light3 and light6=5 minute stimulus duration, 235 minute interstimulus duration). Generally, engineered stem cells exposed to high intensity light proliferated at a higher rate than those stem engineered stem cells exposed to low intensity light. Notably, Myo_FGFR-LOV cells exposed to high or low intensity light proliferated at higher rates than wild-type muscle stem cells exposed to exogenous FGF (“Myo_WT+FGF dark”) at 7 days in suspension. As expected, wild-type muscle stem cells (e.g., not expressing FGFR-LOV) not exposed to exogenous FGF did not proliferate (“Myo_WT−FGF dark”). This data demonstrates that muscle stem cells expressing a fusion protein comprising a light-activatable domain fused to a signaling protein receptor (e.g., FGFR-LOV) are capable of being grown in suspension in response to light under various illumination parameters without the addition of exogenous growth factors (e.g., FGF).

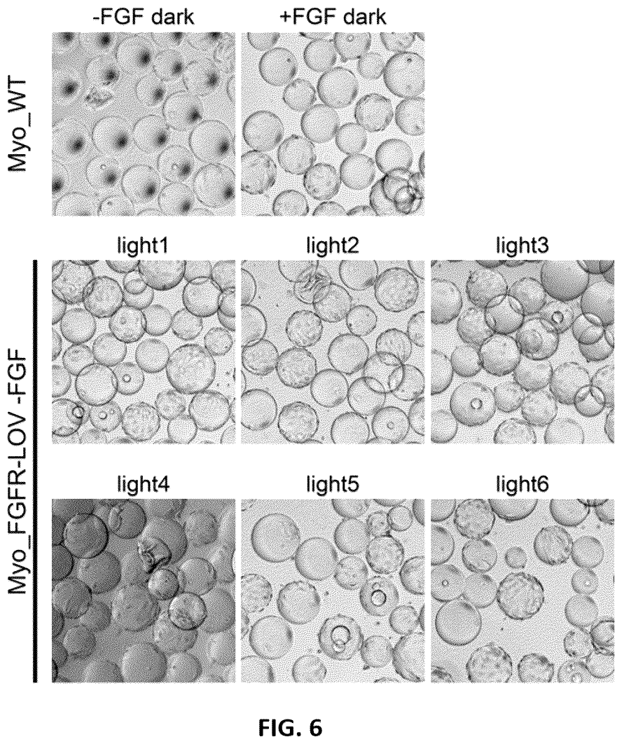

To further demonstrate that muscle stem cells expressing FGFR-LOV proliferate in suspension in response to light, images of the microcarriers were taken. 50 μl of microcarrier suspension was withdrawn from the bioreactor on day 7 of culture, transferred to a well of a 96-well glass bottomed plate (Cellvis, P96-1.5H-N), and the microcarriers were allowed to settle to the bottom of the well. Microcarriers were imaged with a Leica DMi8 Thunder microscope using brightfield illumination and a 5× objective. FIG. 6 demonstrates that muscle stem cells engineered to express FGFR-LOV proliferated for at least 7 days in suspension in response to light using various illumination parameters in the absence of exogenous FGF (“Myo_FGFR-LOV−FGF light1”, “Myo_FGFR-LOV−FGF light2”, “Myo_FGFR-LOV−FGF light3”, “Myo_FGFR-LOV−FGF light4”, “Myo_FGFR-LOV−FGF light5”, and “Myo_FGFR-LOV−FGF light6”). The data further demonstrates that positive control wild-type muscle stem cells exposed to exogenous FGF also proliferated for at least 7 days in suspension (“Myo_WT+FGF dark), whereas wild-type muscle stem cells not exposed to exogenous FGF did not proliferate (“Myo_WT−FGF dark”).

Maintenance of sternness was also assessed in these cell lines. After 7 days of growth in suspension, cells were detached from microcarriers using trypsin, plated at a density of 15,000 cells/cm², and incubated at 37° C. overnight in growth media containing DMEM-F12, 10% FBS, and 1% penicillin/streptomycin/amphotericin B. The following day, cells were fixed for 20 minutes in cold 4% PFA, permeabilized using 0.2% Triton X-100, and blocked in 1% BSA (Sigma, A6003-5G), and 10% FBS. After blocking, cells were stained overnight at 4° C. with a panel of antibodies against transcription factors and surface antigens commonly used to assess stem cell identity including Pax7 (DSHB, AB_528428), Pax3 (DSHB, AB_528426), and CD29 (Biolegend, 303007), as well as controls with no primary antibody. All primary antibodies were used at a final concentration of 5 μg/mL. After overnight incubation, primary antibodies were washed off, and cells were stained with an Alexa Fluor 555-conjugated secondary antibody (ThermoFisher A32773, 1p g/mL) and Hoechst 33342 for 1 hour at room temperature. After one hour, secondary antibodies were washed off, and cells were imaged using a Leica DMi8 Thunder fluorescence microscope. For signal quantification and normalization, cell nuclei were segmented in the Hoechst channel using the Otsu global thresholding method. Total intensity in the 555 channel was measured, the total intensity of the no primary antibody control was subtracted, and the remainder normalized to the segmented cell number.

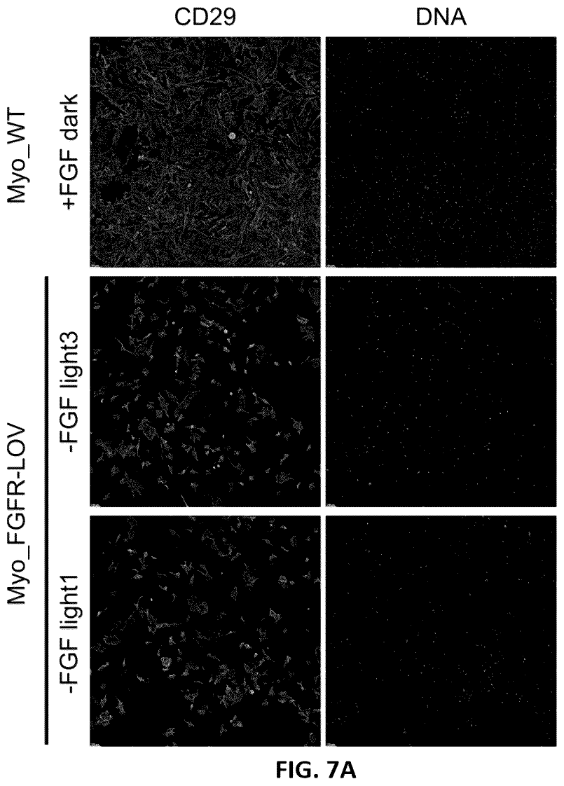

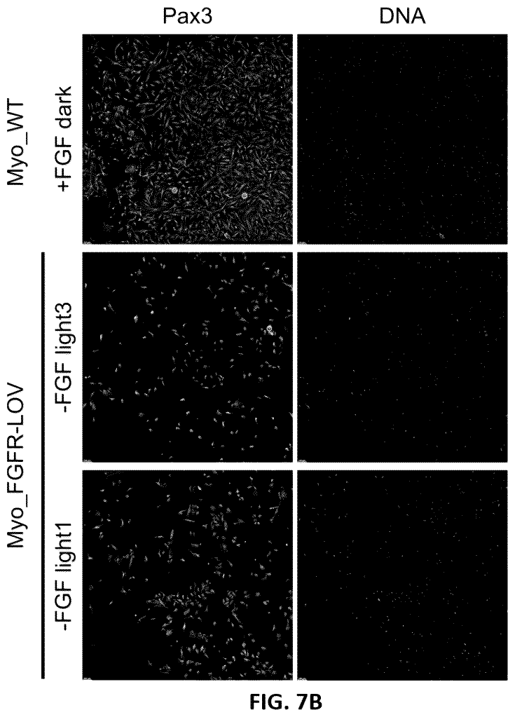

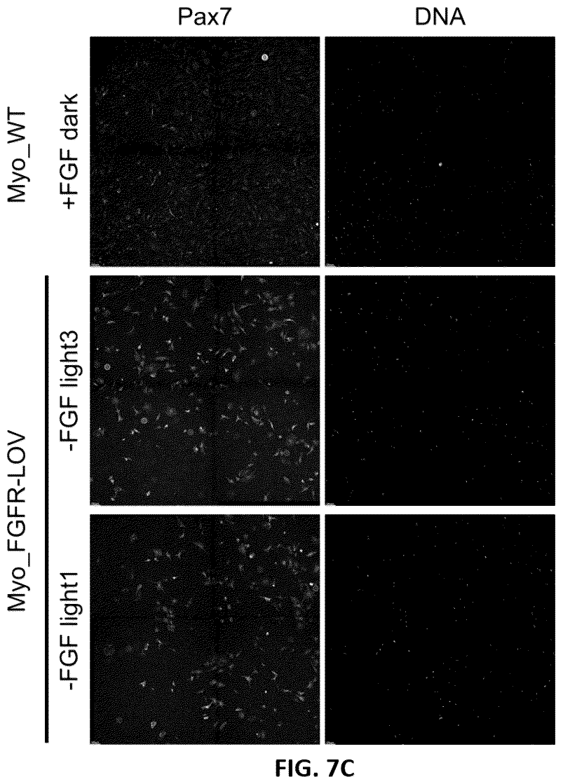

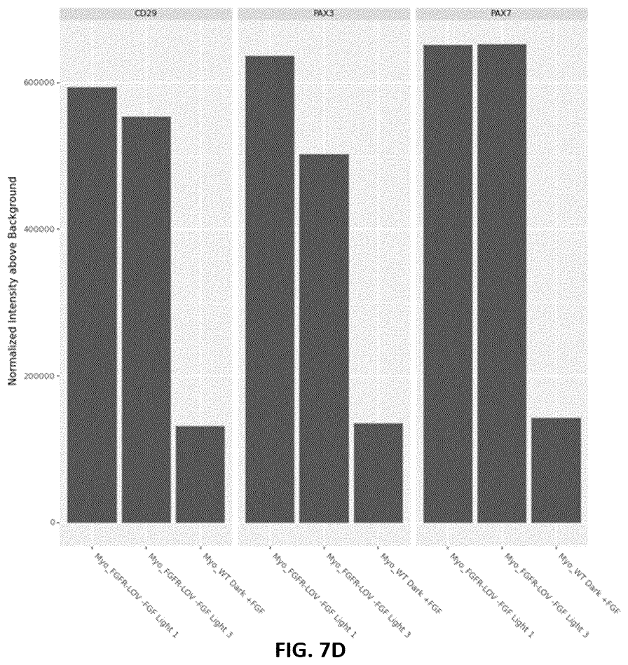

FIGS. 7A-7C show that muscle stem cells expressing FGFR-LOV (“Myo_FGFR-LOV”) expressed the stem cell markers CD29 (FIG. 7A), Pax3 (FIG. 7B), and Pax7 (FIG. 7C), after 7 days in suspension culture with light using various illumination parameters in the absence of exogenous FGF. This data demonstrates that these cells expressed multiple stem cell markers, suggesting that stemness was maintained in these cells after 7 days in suspension culture with light. FIG. 7D depicts a quantification of the signal in the images on FIGS. 7A-7C. The total signal in the antibody channel was normalized to the number of cells, and the normalized signal from the “no primary antibody” conditions was subtracted.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-72. (canceled)

73. A method for growing and/or maintaining eukaryotic cells in suspension, the method comprising:
(a) culturing at least one eukaryotic cell in a culture media in suspension, wherein the at least one eukaryotic cell is genetically engineered to express a fusion protein comprising a signaling protein receptor or a functional portion thereof, and a light-activatable domain; and
(b) exposing the at least one eukaryotic cell to light to activate the light-activatable domain resulting in activation of a downstream signaling pathway of the signaling protein receptor, such that the at least one eukaryotic cell is grown and/or maintained in suspension.

74. The method of claim 73, wherein the signaling protein receptor is a growth factor receptor.

75. The method of claim 73, wherein the culture media is deficient in one or more factors required for growing and/or maintaining the at least one eukaryotic cell, and wherein the one or more factors comprises a ligand for the signaling protein receptor.

76. The method of claim 75, wherein the one or more factors comprises one or more growth factors.

77. The method of claim 76, wherein the one or more growth factors comprises a fibroblast growth factor (FGF).

78. The method of claim 77, wherein the signaling protein receptor is a fibroblast growth factor receptor (FGFR).

79. The method of claim 73, wherein the at least one eukaryotic cell is a mammalian cell.

80. The method of claim 73, wherein the at least one eukaryotic cell is a bovine cell or a chicken cell.

81. The method of claim 73, wherein the at least one eukaryotic cell is a stem cell.

82. The method of claim 81, wherein the stem cell is a multipotent stem cell or a pluripotent stem cell.

83. The method of claim 73, wherein the at least one eukaryotic cell is maintained or is capable of being maintained in suspension for at least 7 days, the at least one eukaryotic cell of the plurality of eukaryotic cells is grown or is capable of being grown in suspension for at least 7 days, or both.

84. The method of claim 81, wherein, at 7 days or greater in suspension culture, the stem cell expresses one or more markers of stemness.

85. The method of claim 73, wherein the culture media is contained within a bioreactor vessel.

86. The method of claim 85, wherein the bioreactor vessel has a total volume of at least 100 milliliters (mL).

87. The method of claim 73, wherein the light-activatable domain is selected from the group consisting of: a Light-Oxygen-Voltage (LOV) photoreceptor domain, a Cryptochrome (CRY) photoreceptor domain, Blue-light-using FAD (BLUF) photoreceptor domain, a Phytochrome (PHY) photoreceptor domain, a cryptochrome-interacting basic-helix-loop-helix protein 1 (CIB1) domain, a phytochrome interacting factor (PIF) domain, a Dronpa domain, a UVR8 photoreceptor domain, a COP1 domain, a BphP1 domain, a QPAS-1 domain, a cobalamin-binding domain (CBD), and any combination thereof.

88. The method of claim 73, wherein the exposing of (b) comprises exposing the at least one eukaryotic cell to light having one or more illumination parameters.

89. The method of claim 88, wherein the one or more illumination parameters comprises light intensity, a temporal pattern of illumination, or both.

90. The method of claim 89, wherein the light intensity is between 1-10 μW/mm².

91. The method of claim 89, wherein the temporal pattern comprises a light stimulus duration of at least about 5 minutes, an interstimulus duration of from about 20 minutes to about 250 minutes, or both.

92. The method of claim 73, wherein the fusion protein, after the exposing to light of (b), dimerizes or oligomerizes.

93. A system for growing and/or maintaining eukaryotic cells in suspension, the system comprising:
(a) a bioreactor vessel comprising a culture media;
(b) at least one eukaryotic cell in suspension in the culture media, wherein the at least one eukaryotic cell has been genetically engineered to express a fusion protein comprising a signaling protein receptor or a functional portion thereof, and a light-activatable domain; and
(c) one or more light source for exposing the at least one eukaryotic cell to light to activate the light-activatable domain resulting in activation of a downstream signaling pathway of the signaling protein receptor or functional portion thereof,
such that the at least eukaryotic cell is grown and/or maintained in suspension.
